(19) United States
(12) Patent Application Publication
(10) Pub. No.: US 2021/0077362 A1
(43) Pub. Date: Mar. 18, 2021

(54) COMPOSITIONS COMPRISING A FATTY PHASE AND AN AQUEOUS PHASE IN THE FORM OF SOLID SPHERES

(71) Applicant: CAPSUM, Marseile (FR)

(72) Inventors: Laurence REHAULT, Orleans (FR); Mathieu GOUTAYER, Saint Malo (FR)

(21) Appl. No.: 16/494,646

(22) PCT Filed: Mar. 16, 2018
(86) PCT No.: PCT/EP2018/056755

§ 371 Date: Sep. 16, 2019

(30) Foreign Application Priority Data
Mar. 17, 2017 (FR) 17 52208

(51) Int. Cl. A61K 8/02; A61K 8/92; A61K 8/25; A61K 8/9789; A61K 8/9717; A61K 8/898; A61K 8/891; A61K 8/37; A61Q 1/10

(52) U.S. Cl. CPC A61K 8/025; A61K 8/92; A61K 8/25; A61K 8/9789; A61K 8/9717; A61K 8/927; A61K 2800/24; A61K 8/891; A61K 8/37; A61Q 1/10; A61K 2800/48; A61K 2800/651; A61K 2800/33; A61K 8/898

(57) ABSTRACT

A composition, in particular a cosmetic composition, comprising a fatty phase and an aqueous phase, the aqueous phase being substantially immiscible with the fatty phase, at ambient temperature and atmospheric pressure, wherein: the aqueous phase is in the form of spheres (S1) that are solid at ambient temperature and atmospheric pressure, comprising at least one hydrophilic gelling agent, which is preferably temperature-sensitive, and the fatty phase comprises at least one lipophilic agent having a suspending capacity, which is preferably thixotropic, preferably a hydrophobic silica.

The object of the present invention relates to compositions, in particular cosmetic compositions, comprising a fatty phase and an aqueous phase, the said aqueous phase being in the form of solid spheres. It also relates to the cosmetic use of the said compositions, in particular for makeup and/or care products for keratin materials, in particular the lips and/or eyelids.

A recurrent problem with cosmetic compositions of such types as glosses (or lip gloss), lipsticks, eyeliners, and eye gloss is their property of tackiness and/or their poor capacity to hydrate or moisturise the lips or the eyelids continuously. This is due to their predominantly anhydrous nature. In effect, it is difficult to maintain the water in a stabilised form in such compositions.

Also, conventional compositions of such types as lip glosses, lipsticks, eyeliners and eye gloss, which impart a high degree of glossy lustre to the surface of the lips or eyelids, necessitate the presence of silicone fluids in the composition. Silicone fluids are known for their high refractive indices which provide glossy lustre. However, in the light of their poor environmental profiles and given that they are relatively expensive, there is a desire to break away from the practice of deploying these types of silicone fluids,

Thus at this time there is a need for new compositions, that have both a high degree of glossiness/lustre properties and satisfactory capacities in respect of hydration, freshness and comfort on application, and preferably free of silicone oils.

The present invention serves the object of providing a composition, in particular a cosmetic composition of such type as glosses, lipsticks, concretes, eyeliners, and eye glosses, that have both a high degree of glossy lustre and satisfactory capacities in respect of hydration, freshness and comfort on application, and preferably free of silicone oils.

The present invention also serves the object of providing a composition, in particular a cosmetic composition of such type as glosses, lipsticks, concretes, eyeliners, and eye glosses, that have both a satisfactory degree of glossy lustre while also being capable of imparting good staying power (performance) over time with minimal tackiness, and good capacities in respect of hydration, as well as freshness and comfort on application.

The present invention also serves the object of providing such a composition that is free of silicone oil.

The present invention also serves the object of providing a composition, in particular a cosmetic composition, in particular of such type as glosses, lipsticks, eyeliners, and eye glosses, that exhibits an immediate hydrating effect upon application, and is able to last up to more than six hours after application.

The present invention also serves the object of providing a composition that is used for stabilising over time an aqueous phase dispersed in a fatty phase without necessarily resorting to conventional stabilising systems, for example of such types as membrane, shell or coacervate, at the interface between the fatty phase and the aqueous phase.

According to one particular embodiment, the present invention also serves the object of providing a composition, in particular a cosmetic composition, and in particular of such type as glosses, lipsticks, eyeliners, and eye glosses, comprising a fatty phase that is transparent or at the very least translucent.

Thus, the present invention relates to a composition, in particular a cosmetic composition, comprising a fatty phase and an aqueous phase, the said aqueous phase being substantially immiscible with the fatty phase, at ambient temperature and at atmospheric pressure, wherein:
the aqueous phase is in the form of spheres (S1) that are solid at ambient temperature and atmospheric pressure, comprising at least one hydrophilic gelling agent, which is preferably temperature-sensitive; and
the fatty phase comprises at least one lipophilic agent having suspending capacity, preferably being thixotropic, preferably a hydrophobic silica.

According to the invention, an agent is said to be thixotropic if under constant stress (or velocity gradient), the apparent viscosity of the phase comprising the said agent decreases over time. Thus the physical property of thixotropy is characterised as follows:
left under prolonged rest conditions, the thixotropic phase will be restructured (its viscosity increases);
under stress that is sufficiently high to break the structure formed at/under rest, the phase can flow and become deconstructed. Its viscosity decreases with the progression of the destructuring.

A composition according to the invention is thus present in form of a dispersion of spheres (S1) in the fatty phase.

A composition according to the invention therefore comprises an aqueous phase in a continuous fatty phase, the aqueous phase not being miscible with the fatty phase, at ambient temperature (for example, T=25° C.±2° C.) and atmospheric pressure (760 mm Hg, that is 1,013·10⁵ Pa or 1013 mbar).

It was found in a surprising manner that the compositions according to the invention combine satisfactory properties in terms of gloss and durability over time as well as good hydration (immediate hydration), freshness and comfort on application on keratinous materials (in particular less sticky and non-impeding). In addition, the hydration property is all the more interesting because it acts Immediately upon application with the effect lasting up to more than 6 hours after application.

Thus, a composition according to the invention is a new alternative for stabilising over time an aqueous phase dispersed in a fatty phase without necessarily resorting to conventional stabilising systems, for example of such types as membrane, shell, coacervate, at the interface between the fatty phase and the aqueous phase.

The stability over time of the spheres (S1) is all the more novel and of beneficial interest when they are macroscopic. When the spheres (S1) are macroscopic, a visual differentiation is obtained, in particular in the field of cosmetic compositions of such types as glosses, lipsticks, eyeliners and eye glosses.

According to one embodiment, at ambient temperature, that is to say at a temperature equal to 25° C.±2° C., the composition according to the invention is a macroscopically homogeneous mixture.

According to the invention, it is the combination of the hydrophilic gelling agent with/and the lipophilic agent having suspending capacity which makes it possible to stabilise the composition according to the invention, and in particular to prevent and/or to avoid the coalescing of the spheres (S1) with each other and creaming of the spheres (S1) in the fatty phase.

A composition according to the invention is preferably free of surfactant. These compositions therefore differentiate themselves from the usual cosmetic compositions.

A composition according to the invention is a topical composition, with administration/application/use therefore being non-oral. Preferably, a composition according to the invention is not a food composition.

Preferably, a composition according to the invention is translucent, or indeed even transparent.

The property of transparency or translucency of the composition according to the invention is determined in the following manner: the composition to be tested is made to flow/poured into a 30 ml Volga jar/pot, the composition is left to stand for a period of 24 hrs at ambient temperature and to be placed there-under, is a white sheet, on which is drawn with a black marker a cross of about 2 mm In thickness. If the cross is visible to the naked eye in the daylight at a viewing distance of 40 cm, the composition is transparent or translucent.

This transparent or translucent appearance is very satisfying, especially for the consumer, from an aesthetic point of view and can, therefore, be of great commercial interest.

Viscosity

The viscosity of the compositions according to the invention may vary in a significant fashion, thereby making it possible to obtain varying textures.

In particular the viscosity spectrum that may be attained is such that a composition according to the invention specifically intended for makeup and/or care of keratin materials, in particular the lips and/or the eyelids may be a lip gloss, a lipstick, an eyeliner and an eye gloss.

According to one embodiment, the composition according to the invention has a viscosity comprised between 1 mPa·s and 500,000 mPa·s, preferably between 10 mPa·s and 300,000 mPa·s and preferably between 1000 mPa·s and 100,000 mPa·s, as measured at 25° C.

In particular, a composition according to the invention, of a gloss type, has a viscosity comprised between 1000 mPa·s and 20,000 mPa·s, preferably between 2,000 mPa·s and 15,000 mPa·s, and more preferably between 5000 mPa·s and 10,000 mPa·s, as measured at 25° C.

Preferably, a composition according to the invention, of a gloss type, has a viscosity that is lower than 20,000 mPa·s, preferably lower than 15 000 mPa·s, more particularly lower than or equal to 10,000 mPa·s.

The viscosity is measured at ambient temperature, for example T=25° C.±2° C. and at ambient pressure, for example 1013 mbar, by the method described here below.

Use is made of a Brookfield type viscometer, typically a Brookfield digital viscometer model RVDV-E (spring torque of 7187.0 dyne-cm), which is a rotational viscometer operating under imposed rotational speed, provided with a spindle rotating element (referred to by the accepted term “Spindle”). A rotational speed is imposed on the rotatable spindle and the measurement of the torque exerted on the spindle makes it possible to determine the viscosity by knowing the geometric parameters/shape of the spindle used.

Use is made for example, of a spindle of size No. 05 (Brookfield reference: RV5). The shear rate corresponding to the measurement of the viscosity is defined by the spindle used and the rotational speed thereof.

The measurement of viscosity is performed over 1 minute at ambient temperature (T=25° C.±2° C.). about 150 g of solution is placed in a beaker of 250 ml volume, with a diameter of about 7 cm in a manner such that the height of the volume occupied by 150 g of solution is sufficient to reach the gauge marked on the spindle. Then, the viscometer is started and run on a speed of 10 revolutions/min and it is necessary to wait for the value displayed on the display screen to be stable. This measure gives the viscosity of the fluid being tested, as mentioned in the context of the present invention.

Aqueous Phase

In accordance with the invention, the compositions according to the invention comprise an aqueous phase in the form of solid spheres (S1) at ambient temperature and at atmospheric pressure.

As indicated here above, the ambient temperature corresponds to a temperature of 25° C.±2° C., and the atmospheric pressure corresponds to a pressure of 1013 mbar.

The spheres (S1) are preferably flexible solid spheres. According to the invention, the term “flexible solid” in particular is used to indicate the fact that the spheres (S1) of the invention do not flow under their own weight, but can be deformed by exerting pressure, for example with a finger. Thus, the consistency thereof is quite similar to that of a butter (without the fatty nature), with a malleable and prehensile (graspable) nature. The spheres (S1) may be spread easily by hand, in particular over a keratin material, in particular on the skin.

Preferably a flexible solid sphere (S1) according to the invention satisfies at least one of the physicochemical criteria 1, 2.a, 2.b, 2.c, and 2.d here below, in particular at least two criteria, preferentially at least three criteria, more preferably at least four criteria, indeed even more preferentially all five criteria 1, 2.a, 2.b, 2.c, and 2.d, developed based on a bulk aqueous phase used for manufacturing the said spheres (S1). Unless otherwise indicated, these criteria shall be measured at ambient temperature (25° C.) and atmospheric pressure (1 atm).

Criterion 1: The aqueous phase according to the invention has a viscoelastic curve at 25° C., measured between 10⁻² Hz and 100 Hz, such that there is no point of intersection between the curves G′ and G″; G′ is always strictly greater than G″ (for measurements carried out at a frequency of between 10⁻² Hz and 100 Hz). The viscoelastic curve is established by means of a Bohlin Gemini imposed stress rheometer in parallel plate (plane-plane) measurement geometry. The temperature was regulated and controlled by a Peltier-effect plane (plate) and an anti-evaporation device (solvent trap filled with water for the measurements performed at 25° C.). Measurements were carried out with oscillation between 10-2 Hz and 100 Hz, at a stress of 1% with a striated P40 plane. The stress of 1% was determined by performing scanning with amplitude so as to be situated within the linear range.

Measurement is performed of G′, which corresponds to the storage modulus reflecting the elastic response and the solid nature of the sample; measurement is performed of G″, which corresponds to the loss modulus reflecting the viscous response and the liquid nature of the sample.

Criteria 2.a to 2.d: the aqueous phase according to the invention is such that it presents:

2.a) a firmness (in g) of less than 400 g, preferably less than 300 g, or indeed even less than 200 g, in particular comprised between 10 g and 400 g, and more preferably between 100 g and 300 g. The firmness is the maximum force measured during the phase of compression (descent) of the probe into the product. In general the maximum force is reached at the moment when the product breaks. That is why the firmness may be referred to as breaking force;

2.b) a breaking strength (in g·s) of less than 1500 g·s, preferably less than 1000 g·s, or indeed even less than 800 g·s, and more preferably less than 500 g·s, in particular comprised between 100 g·s and 1500 g·s, and more preferably between 250 g·s and 1100 g·s. The breaking strength corresponds to the area under the curve Force=f(time) between the time instant when the probe touches the surface of the product and the time instant when the maximum firmness is measured;

2.c) a stress energy (g.$) less than 1500 g·s, preferably less than 1000 g·s, or indeed even less than 500 g·s, and more preferably less than 300 g·s, in particular comprised between 10 g·s and 1500 g·s, and more preferably between 100 g·s and 1300 g·s The stress energy corresponds to the area under the curve Force=f(time) between the time instant when the maximum firmness is reached and the time instant when the probe Is removed from the product; and or

2.d) a tackiness (stickiness) (g) that is less than or equal to 25 g, preferably less than 15 g, and more preferably less than 10 g, in particular comprised between 1 g and 25 g, and more preferably between 5 g and 15 g. The tack corresponds to the maximum force measured during the phase of withdrawal (ascent) of the probe from the product.

The measurements of firmness, breaking strength, stress energy and tack were carried out with a TA.XT Stable Micro Systems texture analyser with the following parameters:
Procedure: Cylindrical, finger-shaped probe made of Teflon (P/0.5 HS),
10 mm penetration,
Speed 1 mm/s,
Trigger force=2 g, and
Measurement performed in 30 mL perfume jars at 20° C.

According to one embodiment, the solid spheres (S1) are full.

According to one other embodiment, the solid spheres (S1) comprise at least one, preferably one single, internal droplet of a liquid composition at ambient temperature, as described further here below.

According to one embodiment, a composition according to the invention is prepared by operational implementation of a “non-microfluidic” method, that is to say, by means of simple emulsification. The size of the spheres (S1) is thus then less than 500 μm, or indeed even less than 200 μm. Preferentially, the size of the spheres (S1) is comprised between 0.5 μm and 50 μm, preferably between 1 μm and 20 μm.

According to this embodiment, the composition according to the invention comprises spheres (S1) of reduced size, in particular as compared to the spheres (S1) obtained by means of a microfluidic method. This small size will have an effect on the texture. Indeed, a composition according to the invention, formed of spheres (S1) that are finely dispersed, is found to exhibit improved properties of unctuousness or creaminess.

According to one other embodiment, a composition according to the invention is prepared by operational implementation of a “microfluidic” method, in particular as has been described here below. According to this embodiment, the size of the spheres (S1) is macroscopic, that is to say, visible to the naked eye, in particular greater than 500 μm, or indeed even greater than 1000 μm. Preferentially, according to this embodiment, the size of the spheres (S1) is comprised between 500 μm and 3000 μm, preferably between 1000 μm and 2000 μm.

In this regard, it was not obvious that the compositions comprising such spheres (S1) having a size greater than 500 μm are stable.

In the context of the present invention, the term “size” is used to refer to the diameter, in particular the average diameter, of the droplets.

A composition according to the invention of such types as lip glosses/eye glosses, manufactured by a microfluidic method, has lower viscosities than those for conventional liquid lip glosses/eye glosses (ie in order to remain compatible with the microfluidic device). However, this lower viscosity does not affect the staying power (durability) over time on the keratin materials, in particular the lips or the eyelids, of a composition according to the invention, and in particular does not affect the durability of the glossy lustre. On the contrary, this enhances the glide-on comfort upon application and the delicate smoothness of the film on the keratin materials.

In addition, the compositions of the invention exhibit a sensorially appealing quality that is unique and different from a microfluidic dispersion stabilised with the use of a coacervate (as described for example in the patent application WO 2012/120043). In fact, the spheres (S1) which may be defined as gelled water beads, have a mechanical strength, more particularly a crushing strength, that is far greater; the user therefore actually feels the beads getting crushed upon application, without it being detrimental to the homogeneity of the composition upon the application.

A composition according to the invention may be referred to as a macroscopically inhomogeneous mixture of two immiscible phases, particularly when the spheres (S1) are macroscopic. In other words, in a composition according to the invention, each of the phases may be adjusted (individualised), in particular with the naked eye.

Preferably, the spheres (S1) are translucent, or indeed even transparent.

Preferably, the spheres (S1) are monodisperse. In the context of the present description, the term “monodisperse spheres” is used to indicate the fact that the population of the spheres according to the invention has a uniform size distribution.

In view of the foregoing, the spheres (S1) of a composition according to the invention are free of shell or membrane, in particular free of polymeric membrane or membrane formed by interfacial polymerisation. In particular, the spheres (S1) of a dispersion according to the invention are not stabilised with the help of a coacervate (type: anionic polymer (carbomer)/cationic polymer (amodimethicone)).

In other words, the contact between the aqueous phase and the fatty phase is direct, without being detrimental to the stability of the composition according to the invention.

The aqueous phase of the compositions of the invention comprises water, and this in a content preferably between 5% and 99% by weight in relation to the weight of the aqueous phase.

In addition to distilled or deionised water, a water that is suitable for the invention may also be a water from a natural source or a floral water.

According to one embodiment, the aqueous phase represents at least 1%, in particular at least 3%, preferably at least 5%, and more preferably at least 10%, by weight in relation to the total weight of the composition.

Preferably, the content by weight of aqueous phase is comprised between 1% and 30%, in particular between 1.5% and 20%, in particular between 2% and 10%, preferably between 3% and 7%, and preferentially between 4% and 6%, by weight in relation to the total weight of the said composition.

According to one particular embodiment, the spheres (S1) of a dispersion according to the invention are stabilised with the use of a coacervate at the interface between the aqueous phase and the fatty phase, in which case the aqueous phase comprises at least one first precursor polymer of the coacervate (anionic type polymer) and the fatty phase comprises at least one second precursor polymer of the coacervate (cationic type polymer).

The formation of the coacervate between these two polymers is usually caused by a change in the conditions of the reaction medium (temperature, pH, concentration of reactants, etc). The coacervation reaction results in the neutralisation of these two charged polymers of opposite polarities and makes possible the formation of a membrane structure by electrostatic interactions between the anionic polymer and the cationic polymer. The membrane thus formed around each sphere typically forms a shell which completely encapsulates the core of the sphere and thus isolates the core of the sphere from the fatty phase.

In the context of the present description, the term “anionic polymer” (or “anionic type polymer”) is understood to refer to a polymer that includes chemical functional groups of the anionic type. The discussion may also include anionic polyelectrolyte.

By way of examples of anionic type polymers, mention may be made of any polymer formed by the polymerisation of monomers of which at least one part bears chemical functional groups of the anionic type, such as carboxylic acid functional groups. Such monomers are for example acrylic acid, maleic acid, or any ethylenically unsaturated monomer having at least one carboxylic acid functional group. It may for example be an anionic polymer comprising monomer units that include at least one carboxylic acid type chemical functional group.

Preferably the anionic polymer is hydrophilic, that is to say soluble or dispersible in water.

Among the examples of anionic polymers that may be appropriate for the operational implementation of the invention, mention may be made of the copolymers of acrylic acid or maleic acid and other monomers, such as acrylamide, alkyl acrylates, C₅-C₈ alkyl acrylates, C₁₀-C₃₀ alkyl acrylates, C₁₂-C₂₂ alkyl methacrylates, methoxypolyethylene glycol methacrylates, hydroxyester acrylates, acrylate crosspolymers, and the mixtures thereof.

According to one embodiment, the anionic polymer according to the invention is a carbomer or cross-linked acrylates/C₁₀-C₃₀ alkyl acrylate copolymer. Preferably, the anionic polymer according to the invention is a carbomer.

In the context of the invention, and unless otherwise indicated, the term “carbomer”, is understood to refer to an optionally cross-linked homopolymer, resulting from the polymerisation of acrylic acid. It is therefore a poly (acrylic acid) optionally crosslinked.

Among the carbomers of the invention, mention may be made of those marketed under the trade name Tego® Carbomer 340FD of Evonik, Carbopol® 981 of Lubrizol, Carbopol ETD 2050 of Lubrizol, or Carbopol Ultrez 10 of Lubrizol.

According to one embodiment, the term “carbomeric” or “carbomer” or “Carbopol®” is understood to refer to an acrylic acid polymer of high molecular weight cross-linked with allyl sucrose or allyl ethers of pentaerythritol (Handbook of Pharmaceutical Excipients, 5^{th} Edition, pill). For example, it involves Carbopol®10, Carbopol®934, Carbopol®934P, Carbopol 940®, Carbopol®941, Carbopol®71 G, Carbopol®980, Carbopol®971P, or Carbopol®974P. According to one embodiment, the viscosity of the said carbomer is comprised between 4000 and 60,000 cP at 0.5% w/w.

Carbomers are known by other names: polyacrylic acids, carboxyvinyl polymers or carboxy polyethylenes.

According to the invention, the anionic polymer may also be a cross-linked acrylates/C₁₀-C₃₀ alkyl acrylate copolymer (International Nomenclature of Cosmetic Ingredients (INCI) name: acrylates/C₁₀-C₃₀ alkyl acrylate crosspolymer) as defined here above.

In accordance with the invention, the compositions according to the invention may comprise a carbomer and a crosslinked acrylates/C₁₀-C₃₀ alkyl acrylate copolymer.

In the context of the present application, and unless otherwise mentioned, the term “cationic polymer” (or “cationic type polymer”) is understood to refer to a polymer that includes chemical functional groups of the cationic type. The discussion may also include cationic polyelectrolyte.

Preferably the cationic polymer is lipophilic or fat soluble (liposoluble).

By way of examples of cationic polymers, mention may be made of any polymer formed by the polymerisation of monomers of which at least one part bears chemical functional groups of the cationic type, such as primary, secondary or tertiary amine functional groups.

Among the examples of cationic polymers that may be appropriate for the operational implementation of the invention, mention may be made of amodimethicone, derived from a silicone polymer (polydimethylsiloxane, also known as dimethicone), modified by primary amine and secondary amine functions.

Mention may also be made of the derivatives of amodimethicone, such as for example copolymers of amodimethicone, aminopropyl dimethicone, and more generally the linear or branched silicone polymers containing amino functions.

Mention may be made of the bis-isobutyl copolymer PEG-14/amodimethicone, Bis (C13-15 Alkoxy) PG-Amodimethicone, Bis-Cetearyl Amodimethicone and bis hydroxy/methoxy amodimethicone.

Mention may also be made of the polysaccharide type polymers comprising amine functional groups, such as chitosan or guar gum derivatives (guar hydroxypropyltrimonium chloride).

Mention may also be made of the polypeptide type polymers comprising amine functional groups, such as polylysine.

Mention may also be polyethyleneimine type polymers comprising amine functional groups, such as linear or branched polyethyleneimine.

According to one particularly preferred embodiment, the cationic polymer corresponds to the following formula:

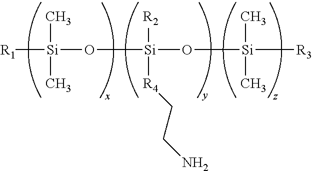

in which:
R₁, R₂ and R₃ independently of one another, represent OH or CH₃;
R₄ represents a group —CH₂— or a group —X—NH— wherein X is a C3 or C4 divalent alkylene radical;
x is an integer occurring between 10 and 5000, preferably between 30 and 1000, and preferably between 80 and 300;
y is an integer occurring between 2 and 1000, preferably between 4 and 100, and preferably between 5 and 20; and
z is an integer occurring between 0 and 10, preferably between 0 and 1, and more preferably is equal to 1.

In the abovementioned formula, when R₄ represents a group —X—NH—, X is connected to the silicon atom. In the abovementioned formula, R₁, R₂ and R₃ represent preferably CH₃. In the abovementioned formula, R₄ is preferably a group —(CH₂)₃—NH—.

Hydrophilic Gelling Agent

A composition according to the invention in addition comprises at least one hydrophilic gelling agent in the aqueous phase, and therefore in the spheres (S1).

According to one embodiment, the hydrophilic gelling agent is selected from the group consisting of natural texturing agents, semi-synthetic texturing agents, synthetic texturing agents, and the mixtures thereof.

By way of texturing agents that are hydrophilic, that is to say soluble or dispersible in water and therefore present in the aqueous phase of a composition according to the invention, mention may be made of:
natural texturing agents, in particular selected from among extracts of algae, exudates of plants, extracts of seeds, exudates of microorganisms, such as alcasealan (INCI: Alcaligenes Polysaccharides), and other natural agents;
semi-synthetic texturing agents, in particular selected from among cellulose derivatives and modified starches;
synthetic texturing agents, in particular selected from among homopolymers of (meth)acrylic acid or one of the esters thereof, copolymers of (meth)acrylic acid or one of the esters thereof, copolymers of AMPS (2-acrylamido-2-methylpropane sulfonic acid), associative polymers;
other texturing agents, in particular selected from among polyethylene glycols (marketed under the trade name Carbowax), clays, silicas such as those marketed under the trade names Aerosil® 90/130/150/200/300/380), glycerine; and
the mixtures thereof.

The term “associative polymer” within the meaning of the present invention, is understood to refer to any amphiphilic polymer comprising in its structure at least one fatty chain and at least one hydrophilic moiety; the associative polymers in accordance with the present invention may be anionic, cationic, nonionic or amphoteric; in particular these are the polymers described in the document FR 2999921. Preferably, they are anionic and amphiphilic associative polymers and nonionic and amphiphilic associative polymers as described here below.

Among the natural texturing agents, mention may particularly be made of extracts of algae represented by the agar-agar, carrageenan, alginates, and the mixtures thereof.

Among the natural texturing agents, mention may particularly be made of exudates of plants represented by tragacanth gum, Karaya gum, Gatty gum, arabic gum, and the mixtures thereof.

Among the natural texturing agents, mention may particularly be made of extracts of seed represented by locust bean gum, guar gum, tara gum, konjac gum, pectins, and the mixtures thereof.

Among the natural texturing agents, mention may particularly be made of exudates of microorganisms represented by xanthan gum, gellan gum, pullulan, and the mixtures thereof.

Among the natural texturing agents, mention may also be made of other natural agents represented in particular by gelatin, collagen, keratin, plant proteins, in particular of wheat and/or soybean, the polymers of chitin or anionic chitosan, cationic, nonionic or amphoteric polymers, hyaluronic acid, or one of the salts thereof, in particular sodium hyaluronate such as that marketed under the trade names HA Oligo, SC Hyaluronic Acid or HyaCare, and the mixtures thereof.

Among the semi-synthetic texturing agents, the cellulose derivatives are in particular represented by carboxymethyl cellulose (CMC) such as that marketed under the trade names Aqualon series or Walocel series; hydroxypropylcellulose (HPC) such as that marketed under the trade name Klucel HPC; hydroxyethylcellulose (HEC) such as that marketed under the trade names Cellosize series or Natrosol 250 series; hydroxyethyl methyl cellulose such as that marketed under the trade name Walocel series; hydroxypropyl methyl cellulose such as that marketed under the trade names Methocel E/F/J/K series from Dow Chemicals, VIVAPHARM CS 152 HV, Benecel E4M, E10M, K100M; methylcellulose such as that marketed under the trade name Methocel A series; ethyl cellulose such as that marketed under the trade name Ethocel series; microcrystalline cellulose such as that marketed under the trade name Avicel PH series; alkylhydroxyethyl cellulose such as cetyl hydroxyethyl cellulose marketed under the trade name Polysurf 67, and the mixtures thereof.

Among the semi-synthetic texturing agents, modified starches are starch derivatives resulting from the modification of the native starch by etherification, esterification or crosslinking, such as in particular sodium carboxymethyl starch such as that marketed under the trade names COVAGEL, VIVASTAR® CS 352 SV ou VIVASTAR CS 302 SV; hydroxypropyl starch such as that marketed under the trade names denominations Zeina B860, Amaze NI, Amycol SQ, Penon PKW; hydroxypropyl starch phosphate such as that marketed under the trade names Structure ZEA/style/XL; and the mixtures thereof.

Among synthetic texturing agents, homopolymers of (meth)acrylic acid or one of the esters thereof are in particular represented by sodium polyacrylates such as those marketed under the trade names Cosmedia SP, Covacryl MV60/MV40, Cosmedia SPL or Luvigel EM; crosslinked polymers of (meth)acrylic acid (or carbomers), such as those marketed under the trade names Carbopol 900 series, Carbopol 2984/5984, Carbopol Ultrez 10/30, particularly Carbopole Ultrez 21, Tego Carbomer 134/140/141, Aqupec HV-505, HV-505HC, HV-504, HV-501, HV-505E, HV-504E, HV-501E, HV-505ED, Ashland 941 carbomer, or Ashland 981 carbomer; and the mixtures thereof. Among these texturing agents, mention may also be made of anionic polymers as mentioned here above, in particular carbomers defined above.

Among synthetic texturing agents, the copolymers of (meth)acrylic acid or one of the esters thereof are in particular represented by glyceryl acrylate/acrylic acid copolymer such as that marketed under the trade names Lubrajel series, Lubrasil series or Norgel; acrylates copolymers such as those marketed under the trade names Carbopol Aqua SF-1 OS Polymer (INCI name=Acrylates copolymer); sodium acrylates crosspolymer-2 such as that marketed under the trade name Aquakeep 10 SH NF; acrylates/C₁₀-C₃₀ alkyl acrylate crosspolymers such as those marketed under the trade name Carbopol 1342/1382, Carbopol ETD 2020, Pemulen TR-1/TR-2, Carbopol Ultrez 20/21, Tego Carbomer 341 ER, Tego Carbomer 750 HD, Tego Carbomer 841 SER, Aqupec HV-501ER, HV-701EDR, HV-501EM, SER W-150C ou SER W-300C; sodium acrylates/beheneth-25 methacrylate crosspolymer such as that marketed under the trade name Novemer EC-2; acrylates/acrylamide copolymers such as that marketed under the trade name Novemer EC-1 by Lubrizol; acrylamide/sodium acrylate copolymers such as that marketed under the trade name Aquagel 55; Acrylic Acid/VP crosspolymers such as that marketed under the trade name Ultrathix P-100; and the mixtures thereof.

Among synthetic texturing agents, copolymers of AMPS are in particular represented by the AMPS NH4/Vinylpyrrolidone copolymers such as that marketed under the trade name Aristoflex AVC (INCI: Ammonium Acryloyldimethyltaurate/VP Copolymer); the AMPS NH4/Beheneth-25 methacrylate copolymers such as that marketed under the trade name Aristoflex HMB (INCI: Ammonium Acryloyldimethyltaurate/Beheneth-25 methacrylate Crosspolymer); AMPS Na/Vinylpyrrolidone copolymers such as that marketed under the trade name Aristoflex AVS (INCI: Sodium Acryloyldimethyl taurate/VP Copolymer); AMPS NH4/2-Carboxyethylacrylate copolymers such as that marketed under the trade name Aristoflex TAC (INCI: Ammonium Acryloyldimethyltaurate/carboxyethyl crosspolymer); AMPS Na/Acrylic acid/Sodium Acrylate/Dimethyl acrylamide copolymers such as that marketed under the trade name Simulgel SMS88 (INCI: Sodium Acrylate/Acryloyldimethyltaurate/Dimethylacrylamide Crosspolymer & Isohexadecane & Polysorbate 60); AMPS Na/Sodium Acrylate copolymers such as those marketed under the trade name Simulgel EG names (INCI: Sodium Acrylate/Sodium Acryloyldimethyl Taurate Copolymer & Isohexadecane & Polysorbate 80) or Simulgel EPG (INCI: Sodium Acrylate/Sodium Acryloyldimethyl Taurate Copolymer & Polyisobutene & Caprylyl/Capryl Glucoside); AMPS Na/Acrylamide copolymers such as those marketed under the trade names Simulgel 600 (INCI: Acrylamide/Sodium Acryloyldimethyltaurate/Isohexadecane/Polysorbate-80) or Sepigel 305 (INCI: Polyacrylamide/C13-C14 Isoparaffin/Laureth-7); AMPS Na/Hydroxyethyl Acrylate copolymers such as those marketed under the trade names Simulgel NS (INCI: Hydroxyethyl Acrylate/Sodium Acryloyldimethyltaurate Copolymer & Squalane & Polysorbate-60), Simulgel INS 100 (INCI: Hydroxyethyl Acrylate/Sodium Acryloyldimethyl Taurate Copolymer & Isohexadecane & Polysorbate 60), Simulgel FL (INCI: Hydroxyethyl Acrylate/Sodium Acryloyldimethyl Taurate Copolymer & Isohexadecane & Polysorbate 60), Sepinov WEO or Sepinov EMT 10 (INCI: Hydroxyethyl Acrylate/Sodium Acryloyldimethyltaurate Copolymer); Acryloyl Dimethyltaurate/Sodium Acrylate/Dimethylacrylamide Crosspolymers such as that marketed under the trade name Sepinov P88 (INCI: Sodium Acrylate/Acryloyldimethyltaurate/Dimethylacrylamide Crosspolymer); and the mixtures thereof.

Among synthetic texturing agents, mention may be made in addition of PVP such as that marketed under the trade name FlexiThix polymer.

Among synthetic texturing agents, amphiphilic and anionic associative polymers are in particular represented by acrylates/Steareth-20 Methacrylate Copolymer such as that marketed under the trade name Aculyn 22; acrylates/Beheneth-25 Methacrylate Copolymer such as that marketed under the trade name Aculyn 28; C₃₀₋₈ Olefin/Isopropyl Maleate/MA Copolymers such as that marketed under the trade name Performa V 1608; Acrylates/Steareth-20 Methacrylate Crosspolymer such as that marketed under the trade name Aculyn 88; Polyacrylate Crosspolymer-6 such as that marketed under the trade name Sepimax Zen; Acrylates/C₁₀-C₃₀ Alkyl Acrylate Crosspolymers such as those mentioned above; and the mixtures thereof.

Among synthetic texturing agents, the nonionic and amphiphilic associative polymers are represented by PEG-150 distearate such as that marketed under the trade name Emanon 3299V; PEG-150/Decyl Alcohol/SMDI Copolymer such as that marketed under the trade name Aculyn 44; PEG-150/stearyl alcohol/SMDI copolymer such as that marketed under the trade name Aculyn 46; acrylates/ceteth-20 itaconate copolymers such as that marketed under the trade name Structure 3001 by AkzoNobel Personal Care; polyurethane polyethers such as those marketed under the trade names Rheolate FX 1100, Rheolate 205, Rheolate 208/204/212, Elfacos T1212, Acrysol RM 184/RM 2020, Adeka Nol GT-700/GT-730; the polyurethane-39 such as that marketed under the trade name Luvigel Star; the cetyl hydroxyethyl cellulose such as those marketed under the trade names Natrosol™ Plus or PolySurf™ 67; and the mixtures thereof.

By way of texturing agents of the aqueous phase, mention may also be made of clays, in particular represented by bentonite such as that marketed under the trade names Veegum, Veegum HS or Vanatural; montmorillonite, hectorite such as that marketed under the trade names Bentone series or Hectone series; kaolinite, and the mixtures thereof.

The composition according to the invention may comprise a single hydrophilic gelling agent as defined here above, or a mixture of at least two hydrophilic gelling agents as defined here above.

A composition according to the invention comprises a sufficient amount of hydrophilic gelling agent(s), that are in particular temperature-sensitive, in order to prevent/limit the phenomena of coalescence of the spheres (S1) with each other. Advantageously, the content of hydrophilic gelling agent(s) is comprised between 0.1% and 15%, preferably between 0.3% and 10%, preferentially between 0.5% and 5%, in particular between 0.8% and 3%, in particular between 1% and 2%, by weight in relation to the weight of aqueous phase of the said composition.

Preferably, the content of hydrophilic gelling agent(s) is comprised between 0.5% and 0.9% by weight in relation to the weight of the aqueous phase of the said composition. This range is particularly advantageous in that it provides an appropriate compromise between the spheres (S1) having good mechanical strength and crushing upon application; this property imparts a particular sensorially appealing quality upon application because the user actually feels the spheres (S1) being crushed upon application.

According to one embodiment, the aqueous phase comprises at least two hydrophilic gelling agents, at least one being a temperature-sensitive hydrophilic gelling agent.

According to the invention, the term “temperature-sensitive hydrophilic gelling agent” is understood to refer to a hydrophilic gelling agent that serves the purpose of increasing the viscosity of the aqueous phase of spheres (S1) that is free of the said gelling agent, the said viscosity evolving in a reversible manner as a function of the temperature.

Thus, a temperature-sensitive hydrophilic gelling agent within the meaning of the present invention is a compound having a melting point above which it is present in liquid form, but below which it is present in solid form and therefore contributes to increasing the viscosity of the phase that comprises the same.

Among these temperature-sensitive hydrophilic gelling agents, mention may be made for example, of gelatin, pectin, agar-agar, and the mixtures thereof.

Preferentially, use is made of agar-agar by way of a temperature-sensitive hydrophilic gelling agent. A preferred composition according to the invention therefore comprises agar-agar by way of a gelling agent. Agar-agar is particularly advantageous in that it has good transparency under cold conditions and a good ratio of “gelation rate/crushing (collapse) upon application”.

According to one preferred embodiment, the content of temperature-sensitive hydrophilic gelling agent(s), in particular of agar-agar, is comprised between 0.1% and 15%, preferably between 0.3% and 10%, preferentially between 0.5% and 5%, in particular between 0.8% and 3%, in particular between 1% and 2%, or indeed even between 0.3% and 0.8%, by weight in relation to the weight of the aqueous phase of the said composition.

Fatty Phase

In accordance with the invention, the compositions according to the invention comprise a fatty phase (or continuous phase) in which solid spheres (S1) mentioned above are dispersed.

The fatty phase according to the invention may represent at least 70%, in particular at least 80%, preferably at least 90%, and more preferably at least 95%, by weight in relation to the total weight of the composition.

According to one embodiment, in the compositions according to the invention, the fatty phase content is comprised between 70% and 99%, preferably between 70% and 95%, in particular between 75% and 90%, and preferentially between 80% and 85%, by weight in relation to the total weight of the said composition.

According to a first variant of the embodiment, the fatty phase is suspensive vis-à-vis the spheres (S1). Thus, the spheres (S1) remain suspended in the fatty phase over a prolonged period of time, for example greater than 1 month, preferably greater than 3 months, and more preferably greater than 6 months. In addition to the associated visual impact, this variant is advantageous in that it makes it possible to prevent/limit the phenomena of coalescence of the spheres (S1) with each other and/or creaming of the spheres (S1) in the fatty phase.

According to a second variant of the embodiment, the fatty phase, is non-suspensive vis-à-vis the spheres (S1). Thus, to the naked eye, a separation of the spheres (S1) from the fatty phase is observed over a period of time of less than 1 month, preferably less than 15 days, more preferably less than 1 week, or indeed even less than 1 day. Such a composition according to the invention is thus then referred to as a two-phase composition. This separation may result from sedimentation or creaming of the spheres (S1) in the fatty phase.

This separation may be immediate after mixing of a composition according to the invention. In other words, the separation between the spheres (S1) and the fatty phase can take place over a period of time between 5 and 60 seconds.

This separation of the two phases of a composition according to the Invention may be brought about over a more prolonged period of time after mixing of a composition according to the invention. In other words, the separation between the spheres (S1) and the fatty phase may take place over a period of time greater than 1 minute, in particular between 1 minute and 300 minutes.

Generally, the separation of the spheres (S1) relative to the aqueous continuous phase is brought about by sedimentation of the spheres (S1), taking into account the hydrophilic nature, and therefore the density thereof which is generally higher than that of the fatty phase.

However, the separation of the spheres (S1) relative to the fatty phase may also be brought about by creaming of the spheres (S1), in which case the person skilled in the art must proceed with the selecting of oil(s) in a manner such that the spheres (S1) have a density that is lower than that of the fatty phase. These selections fall within the domain of general knowledge of the person skilled in the art. For example, it is possible to choose a fluorosilicone type oil (known to have a density greater than 1).

These properties of suspensivity/non suspensivity of the spheres (S1) in the fatty phase are in particular conditioned by the nature and/or the content of oil(s) and/or lipophilic agent(s) having suspending capacity.

Adjustments to be made to the fatty phase, in particular in terms of the nature and/or the content of oil(s) and/or lipophilic agent(s) having suspending capacity vis-à-vis this suspensivity/non suspensivity of the spheres (S1) in the fatty phase fall within the domain of general knowledge of the person skilled in the art in the light of the teaching of the present description.

Advantageously, the fatty phase is shear thinning or pseudoplastic at ambient temperature and atmospheric pressure. The shear thinning, with respect to a fluid, refers to the fact of its “becoming more fluid” when the flow rate increases. More specifically, this means that the dynamic viscosity decreases as the shear rate increases. Also described as shear reducing (shear thinning as per the accepted terminology) or indeed even pseudo-plasticity.

Advantageously, the fatty phase is thixotropic at ambient temperature and atmospheric pressure.

Lipophilic Agent Having Suspending Capacity

A composition according to the invention comprises at least one lipophilic agent having suspending capacity. The term “lipophilic agent having suspending capacity” is understood to refer to an agent capable of increasing the viscosity of the fatty phase in a manner so as to improve the suspensive nature (or power) of the said fatty phase, in particular vis-à-vis the spheres (S1). This lipophilic agent having suspending capacity thus participates in stabilising the composition according to the invention, and in particular in preventing and/or avoiding the phenomena of coalescence of the spheres (S1) with each other and/or the sedimentation or creaming thereof in the fatty phase.

It may also comprise a mixture of at least two lipophilic agents having suspending capacity.

Preferably, the lipophilic agent having suspending capacity according to the invention is selected from among lipophilic gelling agents that are well known to the person skilled in the art, and as detailed here below.

According to one embodiment, the lipophilic gelling agent having suspending capacity is selected from among lipophilic gelling agents, either organic or inorganic, polymeric or molecular; solid fatty substances at ambient temperature and pressure; and the mixtures thereof.

The term “lipophilic gelling agent” within the meaning of the present invention, is understood to refer to a compound capable of gelling the fatty phase of the compositions according to the invention.

The gelling agent is lipo-soluble (fat-soluble) or lipo-dispersible (fat-dispersible).

As is apparent from the sections that follow, the lipophilic gelling agent is advantageously selected from among particulate gelling agents; organopolysiloxane elastomers; semi-crystalline polymers; polyacrylates; esters of sugar/polysaccharide, in particular dextrin esters, inulin esters, glycerol esters; hydrogen bonding polymers; hydrocarbon block copolymers and the mixtures thereof.

Particulate Gelling Agents

The particulate gelling agent used in implementing the composition according to the invention is in particulate form, preferably spherical particles. By way of representative examples of the lipophilic particulate gelling agents that may be appropriate for purposes of the invention, mention may be made most particularly of waxes, either polar and apolar, butters, modified clays, silicas such as fumed silicas and hydrophobic silica aerogels.

The term “wax” considered in the context of the present invention, in a general sense is understood to refer to a lipophilic compound, which is solid at ambient temperature (25° C.), with change in solid/liquid state being reversible, having a melting point that is greater than or equal to 30° C. being able to go up to 200° C. and in particular up to 120° C. Within the meaning of the invention, the melting point temperature corresponds to the temperature of the most endothermic peak observed by thermal analysis (DSC) as described in the standard ISO 11357-3; 1999. The melting point of the wax may be measured by making use of a differential scanning calorimeter (DSC), for example the calorimeter marketed under the trade name/model “MDSC 2920” by the company TA Instruments. The measuring protocol is as follows: A sample of 5 mg of wax placed in a crucible is subjected to a first rise in temperature going from −20° C. to 100° C., at a heating rate of 10° C./minute, then cooled from 100° C. to −20° C. at a cooling rate of 10° C./minute and finally subjected to a second rise in temperature going from −20° C. to 100° C. at a heating rate of 5° C./minute. During the second rise in temperature, measurement is carried out of the variation in the difference in power absorbed by the empty crucible and by the crucible containing the wax sample as a function of temperature. The melting point of the compound is the temperature value corresponding to the tip of the peak of the curve representing the variation of the difference in power absorbed as a function of temperature. The waxes that may be used in the compositions according to the invention are selected from among waxes that are solid at ambient temperature, derived from animal, plant, mineral or synthetic origins, and the mixtures thereof.

The waxes within the meaning of the invention may be those generally used in cosmetics or dermatological fields. In particular they may be polar or non-polar, hydrocarbon-silicone and/or fluorinated waxes, optionally containing ester or hydroxyl functional groups. They may also be of natural or synthetic origin.

The term “apolar wax” within the meaning of this invention is understood to refer to a wax of which the solubility parameter at 25° C. as defined here below, δₐ is equal to 0 (J/cm³)^{1/2}. The definition and calculation of the solubility parameters in the Hansen three dimensional solubility space are described in the article by C M Hansen: “The three dimensional solubility parameters” J. Paint Technol. 39, 105 (1967).

According to this Hansen space:
δ_{D} characterises the London dispersion forces resulting from the formation of dipoles induced during molecular impacts;
δₚ characterises the Debye interaction forces between permanent dipoles as well as the Keesom interaction forces between induced dipoles and permanent dipoles;
δₕ characterises the forces of specific interactions (types of bonds: hydrogen, acid/base, donor/acceptor, etc);
δₐ is determined by the equation: δa=((δₚ²+δₕ²)^{1/2}.

The parameters δₚ, δₕ, δ_{D} and δₐ are expressed in (J/cm³)^{1/2}.

Apolar waxes are in particular hydrocarbon waxes constituted only of carbon atoms and hydrogen atoms and free of heteroatoms such as N, O, Si and P.

The apolar waxes are selected from among microcrystalline waxes, paraffin waxes, ozokerite, polyethylene waxes, and the mixtures thereof. By way of ozokerite mention may be made of Ozokerite Wax SP 1020 P. By way of microcrystalline waxes that may be used, mention may be made of Multiwax W 445® marketed by the company Sonnebom, Microwax HW® and Base Wax 30540® 25 marketed by the company Paramelt and Cerewax No 3 marketed by the company Baerlocher.

By way of microwaxes which may be used in the compositions according to the invention as an apolar wax, mention may be made of polyethylene microwaxes such as those marketed under the trade names Micropoly 200®, 30 220®, 220L® and 2505® by the company Micro Powders. 3025096 34 By way of polyethylene waxes mention may be made of Performalene 500-L Polyethylene and Performalene 400 Polyethylene marketed by New Phase Technologies, Asensa® SC 211 marketed by the company Honeywell.

The term “polar wax” within the meaning of this invention, is understood to refer to a wax of which the solubility parameter at 25° C. δₐ is different from 0 (J/cm³)^{1/2}. In particular, the term “polar wax” is understood to refer to a wax whose chemical structure is formed essentially, indeed even constituted, of carbon atoms and hydrogen atoms, and comprising at least one strongly electronegative heteroatom such as an atom of oxygen, nitrogen, silicon or phosphorus. The polar waxes may in particular be hydrocarbon, fluorinated or silicone waxes. Preferentially, the polar waxes may be hydrocarbon waxes.

The term “hydrocarbon wax” is understood to refer to a wax that is formed essentially, indeed even constituted, of atoms of carbon and hydrogen, and possibly of atoms of oxygen, nitrogen and containing no silicon or fluorine atom. It may contain alcohol, ester, ether, carboxylic acid, amine and/or amide groups.

The term “ester wax”, according to the invention is understood to refer to a wax comprising at least one ester functional group.

The term “alcohol wax” according to the invention is understood to refer to a wax comprising at least one alcohol functional group, that is to say comprising at least one free hydroxyl group (OH).

In particular the following may be used by way of ester wax:
ester waxes, such as those selected from among:

i) waxes having the formula R₁COOR₂ wherein R₁ and R₂ represent linear, branched or cyclic, aliphatic chains, whereof the number of atoms ranges from 10 to 50, which may contain one heteroatom such as O, N or P, and whereof the melting point temperature ranges from 25 to 120° C.;

ii) di-(trimethylol-1,1,1 propane)tetrastearate, marketed under the trade name Hest 2T-4S by the company Heterene;

iii) diester waxes of a carboxylic diacid having the general formula R₃— (—OCOR₄—COO—R₅), wherein R₃ and R₅ are identical or different, preferably identical, and represent a C₄-C₃₀ alkyl group (alkyl group having 4 to 30 carbon atoms), and R₄ represents a C₄-C₃₀ aliphatic group (alkyl group having 4 to 30 carbon atoms) that is linear or branched, and may or may not contain one or more unsaturation(s), and being preferably linear and unsaturated;

iv) Mention may also be made of waxes obtained by catalytic hydrogenation of animal or plant oils containing fatty chains, either linear or branched, having C₈-C₃₂, for example, such as hydrogenated jojoba oil, hydrogenated sunflower oil, hydrogenated castor oil, hydrogenated coconut oil, as well as the waxes obtained by hydrogenation of castor oil esterified with cetyl alcohol;

v) beeswax, synthetic beeswax, polyglycerolated beeswax, carnauba wax, candelilla wax, oxypropylenated lanolin wax, rice bran wax, Ouricury wax, alfalfa wax, cork fibre wax, sugar cane wax, Japan wax, sumac wax, montan wax, Orange wax, Laurier wax, hydrogenated jojoba wax, sunflower wax, lemon wax, olive wax, berry wax.

In a particular manner, mention may be made of C18-C38 FATTY ALCOHOL HYDROXYSTEAROYL STEARATE; INCI name: SYNTHETIC BEESWAX and marketed under the trade name KESTERWAX K82P by the company Koster Keunen.

According to one other embodiment, the polar wax may be an alcohol wax. The term “alcohol wax” is understood according to the invention to refer to a wax comprising at least one alcohol functional group, that is to say comprising at least one free hydroxyl group (OH). By way of an alcohol wax mention may be made, for example of the wax C30-50 Alcohols Performacol 550 Alcohol marketed by the company New Phase Technology, stearic alcohol, cetyl alcohol.

Use may also be made of silicone waxes which may advantageously be substituted polysiloxanes, preferably having a low melting point. The term “Silicone wax” is understood to refer to an oil comprising at least one silicon atom, and in particular comprising Si—O groups. Among the commercial silicone waxes of this type, in particular mention may be made of those marketed under the trade names Abilwax 9800, 9801 or 9810 (Goldschmidt), KF910 and KF7002 (Shin-Etsu), or 176-1118-3 and 176-11481 (General Electric).

The silicone waxes that can be used may also be alkyl or alkoxydimethicones, as well as (C₂₀-C₆₀)alkyl dimethicones, in particular the (C₃₀-C₄₅)alkyl dimethicones such as the silicone wax marketed under the trade name SF-1642 by the company GE-Bayer Silicones or C₃₀₋₄₅ Alkyldimethylsilyl polypropylsilsesquloxane under the trade name SW-8005® C30 Resin Wax marketed by the company Dow Corning.

In the context of the present invention, by way of particularly advantageous waxes, mention may be made of polyethylene waxes, jojoba wax, and silicone waxes.

According to one particular embodiment of the invention, use will be made of the waxes having melting point greater than 45° C. comprising one or more C₄₀-C₇₀ ester compounds and not comprising any C₂₀-C₃₉ ester compound. The term “ester compound” is understood to refer to any organic molecule comprising a linear or branched, saturated or unsaturated, hydrocarbon chain, comprising at least one ester functional group having the formula —COOR wherein R represents a hydrocarbon radical, in particular a linear, saturated alkyl radical. The term “wax not comprising any C₂₀-C₃₉ ester compound”, is understood to refer to any wax containing less than 1% by weight of C₂₀-C₃₉ ester compound, preferably less than 0.5% by weight in relation to the weight of the wax, or indeed even free of C₂₀-C₃₉ ester compound.

The waxes according to the invention can also be used in the form of a mixture of waxes. The ester content comprising from 40 to 70 carbon atoms preferably ranges from 20 to 100% by weight and preferably from 20 to 90% by weight in relation to the total weight of wax(es).

Use will be made more particularly of Candelilla wax and/or beeswax.

Mention may also be made of CRYSTALWAX (INCI: Hydroxystearic Acid (and) Synthetic Wax (and) Triisostearin (and) Polybutene (and) Pentaerythrityl Tetraisostearate) marketed by the company Sensient Cosmetic Technologies.

The term “butter” (also referred to as “pasty fatty substance”) within the meaning of the present invention, is understood to refer to a lipophilic fatty compound with change in solid/liquid state being reversible, and having at a temperature of 25° C. a liquid fraction and a solid fraction, and at atmospheric pressure (760 mm Hg). In other words, the starting melting temperature of the pasty compound may be less than 25° C. The liquid fraction of the pasty compound measured at 25° C. may represent 9% to 97% by weight of the compound. This liquid fraction at 25° C. represents preferably between 15% and 85%, more preferably between 40 and 85% by weight. Preferably, the one or more butter(s) have a melting completion temperature of less than 60° C. Preferably, the one or more butter(s) have a hardness less than or equal to 6 MPa.

Preferably, the butters or pasty fatty substance exhibit in the solid state, an anisotropic crystalline organisation, visible by means of X-ray observations.

Within the meaning of the invention, the melting point temperature corresponds to the temperature of the most endothermic peak observed by thermal analysis (DSC) as described in the standard ISO 11357-3; 1999. The melting point of a pasty substance or a wax may be measured by making use of a differential scanning calorimeter (DSC), for example the calorimeter marketed under the trade name “DSC Q2000” by the company TA Instruments.

With respect to the measurement of the melting temperature and determination of the melting completion temperature, the protocols for preparation of samples and measurement are as follows: A sample of 5 mg of pasty fatty substances (or butter) or of wax preheated to 80° C. and collected under magnetic agitation with the use of a spatula which is also heated and placed in a hermetically sealed capsule made of aluminum, or a crucible. Two tests are performed to ensure reproducible results.

The measurements are performed on the calorimeter mentioned here above. The furnace is subjected to a nitrogen purge. Cooling is provided by the heat exchanger RCS 90. The sample is subsequently subjected to the following protocol while initially being brought to a temperature of 20° C., and thereafter subjected to a first rise in temperature going from 20° C. to 80° C., at a heating rate of 5° C./minute, it is then cooled from 80° C. to −80° C. at a cooling rate of 5° C./minute and finally subjected to a second rise in temperature going from −80° C. to 80° C. at a heating rate of 5° C./minute. During the second rise in temperature, measurement is carried out of the variation in the difference in power absorbed by the empty crucible and by the crucible containing the butter sample as a function of temperature. The melting point of the compound is the temperature value corresponding to the tip of the peak of the curve representing the variation of the difference in power absorbed as a function of temperature. The melting completion temperature corresponds to the temperature at which 95% of the sample has melted.

The liquid fraction by weight of the butter (or pasty fatty substance) at 25° C. is equal to the ratio between the enthalpy of fusion consumed at 25° C. and the enthalpy of fusion of the butter. The enthalpy of fusion of the butter or pasty compound is the enthalpy or heat consumed by the compound in order to change from the solid state to the liquid state.

The butter is the said to be in the solid state when the entirety of its mass is in solid crystalline form. The butter is the said to be in the liquid state when the entirety of its mass is in liquid form. The enthalpy of fusion of the butter is equal to the integral of the entire fusion curve obtained using the above-mentioned calorimeter with a temperature rise of 5° C. or 10° C. per minute according to the standard ISO 11357-3:1999. The enthalpy of fusion of butter is the amount of energy needed to cause the compound to change from the solid state to the liquid state. It is expressed in J/g.

The enthalpy of fusion consumed at 25° C. is the amount of energy absorbed by the sample to change from the solid state to the state it exhibits at 25° C. formed of a liquid fraction and solid fraction. The liquid fraction of the butter measured at 32° C., preferably represents from 30% to 100% by weight of the compound, preferably from 50 to 100%, more preferably from 60 to 100% by weight of the compound. When the liquid fraction of the butter measured at 32° C. is equal to 100%, the temperature of the end of fusion range of the pasty compound is 32° C. or lower. The liquid fraction of the butter measured at 32° C. is equal to the ratio between the enthalpy of fusion consumed at 32° C. and the enthalpy of fusion of the butter. The enthalpy of fusion consumed at 32° C. is calculated in the same manner as the enthalpy of fusion consumed at 23° C.

Regarding the measurement of hardness, the protocols for preparing samples and for measurement are as follows: the composition according to the invention or the butter is placed in a mould 75 mm in diameter which is filled to about 75% of its height. In order to overcome thermal history and to control crystallisation, the mould is placed in a programmable Vôtsch VC0018 oven where it is first of all placed at a temperature of 80° C. for a period of 60 minutes, then cooled from 80° C. to 0° C. at a cooling rate of 5° C./minute, then left at the stabilised temperature of 0° C. for 60 minutes, then subjected to a temperature rise going from 0° C. to 20° C. at a heating rate of 5° C./minute, and subsequently left at the stabilised temperature of 20° C. for 180 minutes. Measurement of the compression force is conducted with the TA/TX2i texture analyzer by Swantech. The spindle used is selected according to texture: —steel cylindrical spindle of 2 mm in diameter for very rigid raw materials; —steel cylindrical spindle of 12 mm in diameter for scarcely rigid raw materials. The measurement comprises 3 steps: a 1st step after automatic detection of the surface of the sample when the spindle moves at a measuring speed of 0.1 mm/s, and enters the composition according to the invention or the butter to a penetration depth of 0.3 mm, the software records the value of the maximum force reached; a 2nd so-called relaxation step, when the spindle remains at this position for one second and where the force is recorded after 1 second of relaxation; finally a 3rd so-called withdrawal step when the spindle returns to its initial position at a rate of 1 mm/s and the withdrawal energy of the probe is recorded (negative force).

The hardness value measured during the first step corresponds to the maximum compressive force measured in Newtons divided by the surface area of the cylindrical probe of the texture analyzer expressed in mm² in contact with the butter or the composition according to the invention. The hardness value obtained is expressed in mega-pascals or MPa.

The pasty fatty substance or butter may be selected from among synthetic compounds and compounds of plant origin, A pasty fatty substance may be obtained by synthesis from starting products of plant origin.

The pasty fatty substance is advantageously selected from among:
lanolin and derivatives thereof such as lanolin alcohol, oxyethylenated lanolins, acetylated lanolin, esters of lanolin such as isopropyl lanolate, oxypropylenated lanolins;
polymer or non-polymer silicone compounds such as polydimethysiloxanes of high molecular weight, polydimethysiloxanes with side chains of alkyl or alkoxy type having 8 to 24 carbon atoms, in particular stearyl dimethicones,
polymer or non-polymer fluorinated compounds;
vinyl polymers, in particular:
olefin homopolymers,
olefin copolymers, homopolymers and copolymers of hydrogenated dienes,
straight-chain or branched oligomers, homo- or copolymers of alkyl (meth)acrylates preferably having a C₆-C₃₀ alkyl group,
homo oligomers and copolymers of vinyl esters having C₈-C₃₀ alkyl groups,
homo oligomers and copolymers of vinyl ethers having C₈-C₃₀ alkyl groups,
liposoluble polyethers resulting from polyetherification between one or more C₂-C₁₀₀ diols, preferably C₂-C₅₀ diols,
esters and polyesters, and
mixtures thereof.

In one preferred embodiment of the invention, the particular butter or butters are of plant origin and such as those described in Ullmann's Encyclopaedia of Industrial Chemistry («Fats and Fatty Oils», A. Thomas, published on 15 Jun. 2000, D01: 10.1002/14356007.a10_173, point 13.2.2.2F. Shea Butter, Borneo Tallow, and Related Fats (Vegetable Butters)).

Mention may be made of more particularly of C₁₀-C₁₈ triglycerides (INCI name: C10-18 Triglycerides) which, at a temperature of 25° C. and at atmospheric pressure (760 mm Hg), comprise a liquid fraction and a solid fraction: shea butter, Shea Nilotica butter (Butyrospermum parkii), Galam butter (Butyrospermum parkii), Borneo butter (or tengkawang tallow) (Shorea stenoptera), Shorea butter, illipe butter, Madhuca or Bassia Madhuca longifolia butter, mowrah butter (Madhuca latifolia), Katiau butter (Madhuca mottleyana), Phulwara butter (M. butyracea), mango butter (Mangifera indica), Murumuru butter (Astrocatyum murumuru), Kokum butter (Garcinia indica), Ucuuba butter (Virola sebifera), Tucuma butter, Painya butter (Kpangnan) (Pentadesma butyracea), coffee butter (Coffee arabica), apricot butter (Prunus armeniaca), Macadamia butter (Macadamia Temifolia), grapeseed butter (Vitis vinifera), avocado butter (Persea gratissima), olive butter (Olea europaea), sweet almond butter (Prunus amygdalus dulcis), cocoa butter (Theobroma cacao) and sunflower seed butter, butter under the INCI name Astrocaryum Murumuru Seed Butter, butter under the INCI name Theobroma grandiflorum Seed Butter, and the butter under the INCI name Irvingia gabonensis Kernel Butter, the esters of jojoba (mixture of hydrogenated jojoba oil and wax) (INCI name: Jojoba esters) and the ethyl esters of shea butter (INCI name: Shea butter ethyl esters), and mixtures thereof.

The composition according to the invention may comprise at least one lipophilic clay. The clays may be natural or synthetic clays and are made lipophilic by treatment with an alkyl ammonium salt such as a C₁₀ to C₂₂ ammonium chloride, for example di-stearyl di-methyl ammonium chloride. They may be selected from among bentonites, in particular hectorites and montmorillonites, beidellites, saponites, nontronites, the sepiolites, biotite, the attapulgites, vermiculites and zeolites.

Preferably they are selected from hectorites. Preferably, by way of lipophilic Gays, use will be made of hectorites modified with a C₁₀-C₂₂ ammonium chloride, such as hectorite modified with di-stearyl di-methyl ammonium chloride, di-methyl ammonium such as, for example, that marketed under the trade name Bentone 38V® by the company Elementis or Bentone gel in isododecane marketed under the trade name Bentone gel ISD V® (Isododecane 87%/Disteardimonium Hectorite 10%/Propylene carbonate 3%) by the company Elementis.

The fatty phase of a composition according to the invention may by way of a gelling agent also comprise a fumed (pyrogenated) silica or silica aerogel particles.

Hydrophobic surface treated fumed silica is particularly suitable for use in the invention. It is possible chemically to modify the surface of silica via chemical reaction generating a reduction in the number of silanol groups on the silica surface. In particular, silanol groups can be substituted by hydrophobic groups: a hydrophobic silica is thereby obtained.

The hydrophobic groups may be:
trimethylsiloxyl groups, which are notably obtained by treating pyrogenated silica in the presence of hexamethyldisilazane. Silicas thus treated are called «Silica silylate» according to the CTFA dictionary (8th Edition, 2000). For example, they are marketed under the references Aerosil R812® by the company DEGUSSA, CAB-O-SIL TS-530® by the company CABOT; or
dimethylsilyloxyl or polydimethylsiloxane groups, which are obtained in particular by treating pyrogenated silica in the presence of polydimethylsiloxane or dimethyldichlorosilane. Silicas thus treated are called «Silica dimethyl silylate» according to the CTFA dictionary (8^{th} Edition, 2000). For example, they are marketed under the references Aerosil R972® and Aerosil R974® by the company DEGUSSA, CAB-O-SIL TS-610® and CAB-O-SIL TS-720® by the company CABOT.

The fatty phase of a composition according to the invention may also comprise as gelling agent at least particles of silica aerogels. Silica aerogels are porous materials obtained by replacing (by drying) the liquid component of a silica gel with air. They are generally synthesised by sol-gel process in a liquid medium and then usually dried by extraction of a supercritical fluid, the most commonly used being the supercritical CO₂. This type of drying makes it possible to prevent the contraction of the pores and of the material. The sol-gel process and the various drying operations are described in detail in Brinker C J., and Scherer G. W., Sol-Gel Science: New York: Academic Press, 1990. The particles of hydrophobic silica aerogels used in the present invention have a specific surface area per unit of mass (SM) ranging from 500 to 1500 m²/g, preferably from 600 to 1200 m²/g and more preferably from 600 to 800 m²/g, and a particle size expressed in volume mean diameter (D[0.5]) comprised between 1 to 1500 μm, better yet from 1 to 1000 μm, preferably from 1 to 100 μm, in particular from 1 to 30 μm, more preferably from 5 to 25 μm, yet more preferably from 5 to 20 μm and indeed ever better yet from 5 to 15 μm.

According to one embodiment, the hydrophobic silica aerogel particles used in the present invention have a size expressed in volume mean diameter (D[0.5]) ranging from 1 to 30 μm, preferably from 5 to 25 μm, more preferably from 5 to 20 μm and indeed ever better yet from 5 to 15 μm. The specific surface area per unit of mass may be determined by the nitrogen absorption method referred to as BET (Brunauer-Emmett-Teller) method described in “The Journal of the American Chemical Society”, vol. 60, page 309, February 1938, and corresponding to the international standard ISO 5794/1 (Annex D). The BET specific surface area corresponds to the total specific surface area of the particles considered. The sizes of the silica aerogel particles can be measured by means of static light scattering making use of a commercial granulometer of the type MasterSizer 2000 from Malvern. The data are processed on the basis of the Mie scattering theory. This theory, which is exact for isotropic particles, provides the means to determine in the case of non-spherical particles, an “effective” diameter particles. This theory is described in particular in the book by Van de Hulst, H C, “Light Scattering by 20 Small Particles”, Chapters 9 and 10, Wiley, New York, 1957.

According to one advantageous embodiment, the hydrophobic silica aerogel particles used in the present invention have a specific surface area per unit mass (MS) ranging from 600 to 800 m²/g.

The silica aerogel particles used in the present invention may advantageously have a tamped density p ranging from 0.02 g/cm³ to 0.10 g/cm³, preferably from 0.03 g/cm³ to 0, 08 g/cm³, in particular ranging from 0.05 g/cm³ to 0.08 g/cm³. In the context of the present invention, this density can be assessed in accordance with the following protocol, referred to as the tamped (or tapped) density: 40 g of powder is poured into a graduated measuring cylinder; then the measuring cylinder is subsequently placed on the device STAV 2003 from Stampf Volumeter; the measuring cylinder is then subjected to a series of 2500 tamping/settling manoeuvres (this operation is repeated until the difference in volume between two consecutive tests is less than 2%); and thereafter measurement is directly performed on the measuring cylinder of the final volume Vf of packed tamped powder. The tamped density is determined by the ratio m/Vf, in the case at hand 40/Vf (Vf being expressed in cm³ and m in g).

According to one preferred embodiment, the hydrophobic silica aerogel particles used in the present invention have a specific surface area per unit volume SV ranging from 5 to 60 m²/cm³, preferably from 10 to 50 m²/cm³, and more preferably from 15 to 40 m²/cm³. The specific surface area per unit volume is given by the relation: Sᵥ=S_{M}×p; where p is the tamped density expressed in g/cm³ and S_{M} is the specific surface area per unit mass expressed in m²/g, as has been defined here above.

Preferably, the hydrophobic silica aerogel particles according to the invention have an oil absorption capacity measured at the Wet Point ranging from 5 to 18 ml/g, preferably from 6 to 15 ml/g and more preferably from 8 to 12 ml/g. The absorption capacity measured at the Wet Point, and denoted Wp corresponds to the amount of oil it is necessary to add to 100 g of particles in order to obtain a homogeneous paste. It is measured according to the so-called Wet Point method or method for determining the oil absorption by the powder as described in the standard NF T 30-022. It corresponds to the amount of oil adsorbed onto the available surface area of the powder and/or absorbed by the powder by measuring the Wet Point, described here below: a quantity m=2 g of powder is placed onto a glass plate and then oil (isononyl isononanoate) is added dropwise thereto. After addition of 4 to 5 droplets of oil into the powder, it is mixed using a spatula and add the addition of oil is continued until obtaining the formation of conglomerates of oil and powder. From this time instant onward, the oil is added one drop at a time and the mixture is thoroughly triturated and mashed with the spatula. The addition of oil is discontinued when a firm and smooth paste is obtained. This paste should be left to spread extending over the glass plate without cracks or formation of lumps. Then the volume Vs (expressed in ml) of oil used should be noted. The oil absorption corresponds to the ratio Vs/m.

The aerogels used according to the present invention are hydrophobic silica aerogels, preferably silylated silica (INCI name: silica silylate).

The term “Hydrophobic silica” is understood to refer to any silica whose surface has been treated with silylating agents, for example, halogenated silanes such as alkylchlorosilanes, siloxanes, in particular dimethylsiloxanes such as hexamethyldisiloxane, or silazanes, in a manner so as to functionalise the OH groups by the silyl groups Si—Rn, for example, trimethylsilyl groups. With regard to the preparation of hydrophobic silica aerogel particles modified at the surface by silylation, reference may be made to the document U.S. Pat. No. 7,470,725. Use will preferably be made of hydrophobic silica aerogel particles modified at the surface by trimethylsilyl groups, preferably those corresponding to the INCI name Silica silylate. By way of a hydrophobic silica aerogels that may be used in the invention mention may be made for example of the aerogel marketed under the trade name VM-2260 or VM-2270 (INCI name: Silica Silylate), by Dow Corning, whose particles have an mean size of about 1000 microns and a surface area per unit mass ranging from 600 to 800 m²/g. Mention may also be made of the aerogels marketed by the company Cabot under the references Aerogel TLD 201, Aerogel OGD 201, Aerogel TLD 203, ENOVA® Aerogel MT 1100. ENOVA Aerogel MT 1200. Use will preferably be made of the aerogel marketed under the trade name VM-2270 (INCI name Silica silylate), by the company Dow Corning, the aerogels having particles with a mean size ranging from 5-15 microns and a specific surface area per unit mass ranging from 600 to 800 m²/g.

Organopolysiloxane Elastomer

The organopolysiloxane elastomer has the advantage of imparting good application properties to the composition according to the invention. It procures a very soft-to-the-touch sensation after application, particularly advantageous for application over the skin. It also serves to enable effectively filling in hollow sections present on the keratin materials.

The term “organopolysiloxane elastomer” or “silicone elastomer” is understood to refer to an organopolysiloxane that is flexible, and deformable having viscoelastic properties and in particular the consistency of a sponge or of a soft sphere. Its modulus of elasticity is such that this material is resistant to deformation and has a limited capacity for extension and contraction. This material is capable of regaining its original form/shape after being stretched. This is more particularly a crosslinked organopolysiloxane elastomer.

Thus, the organopolysiloxane elastomer can be obtained by the crosslinking addition reaction of diorganopolysiloxane containing at least one hydrogen bonded to the silicon and of diorganopolysiloxane containing ethylenically unsaturated groups bonded to silicon, in particular in the presence of a platinum catalyst; or by dehydrogenation crosslinking condensation reaction between a hydroxyl terminated diorganopolysiloxane and a diorganopolysiloxane containing at least one hydrogen bonded to silicon, in particular in the presence of an organotin [compound]; or by crosslinking condensation reaction of a diorganopolysiloxane containing hydroxyl end groups and of a hydrolysable organopolysilane; or by thermal crosslinking of organopolysiloxane, in particular in the presence of an organoperoxide catalyst; or by crosslinking of organopolysiloxane by high-energy radiation such as gamma rays, ultraviolet rays or an electron beam.

Preferably, the organopolysiloxane elastomer is obtained by crosslinking addition reaction (A) of diorganopolysiloxane containing at least two hydrogens each bonded to a silicon, and (B) of diorganopolysiloxane having at least two ethylenically unsaturated groups bonded to silicon, in particular in the presence (C) of platinum catalyst, as described for example in the patent application EP-A-295886.

In particular, the organopolysiloxane elastomer can be obtained by reaction of dimethylpolysiloxane containing dimethylvinylsiloxy end groups and of methylhydrogenpolysiloxane with trimethylsiloxy end groups, in the presence of a platinum catalyst. The compound (A) is the base reagent for the formation of elastomeric organopolysiloxane and the crosslinking is effected by addition reaction of the compound (A) with the compound (B) in the presence of the catalyst (C). The compound (A) is in particular an organopolysiloxane having at least two hydrogen atoms bonded to distinct silicon atoms in each molecule. The compound (A) may have any molecular structure, in particular a straight chain or branched chain structure or a cyclic structure. The compound (A) may have a viscosity at 25° C. ranging from 1 to 50,000 centistokes, in particular in order to have good miscibility with compound (B). The organic groups bonded to the silicon atoms of the compound (A) may be alkyl groups such as methyl, ethyl, propyl, butyl, octyl; substituted alkyl groups such as 2-phenylethyl, 2-phenylpropyl, 3,3,3-trifluoropropyl; aryl groups such as phenyl, tolyl, xylyl; substituted aryl groups such as phenylethyl; and substituted monovalent hydrocarbon groups such as an epoxy group, a carboxylate ester group, or a mercapto group. The compound (A) can thus be selected from trimethylsiloxy-terminated methylhydrogenpolysiloxanes, dimethylsiloxane-methylhydrogensiloxane copolymers containing trimethylsiloxy end groups, dimethylsiloxane-methylhydrogensiloxane cyclic copolymers. The compound (B) is advantageously a diorganopolysiloxane having at least two lower alkenyl groups (for example with C₂-C₄); the lower alkenyl group may be selected from among the vinyl, allyl, and propenyl groups. These lower alkenyl groups may be located in any position of the organopolysiloxane molecule but are preferably located at the ends of the organopolysiloxane molecule.

The organopolysiloxane (B) may have a structure based on a branched chain, straight chain, cyclic chain or network, but the linear chain structure is preferred. The compound (B) may have a viscosity ranging from the liquid state to the gum state. Preferably, the compound (B) has a viscosity of at least 100 centistokes at 25° C. In addition to the aforementioned alkenyl groups, other organic groups bonded to the silicon atoms in the compound (B) may be alkyl groups such as methyl, ethyl, propyl, butyl or octyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon groups such as an epoxy group, a carboxylate ester group or a mercapto group. The organopolysiloxane (B) can be selected from among methylvinylpolysiloxanes, methylvinylsiloxane-dimethylsiloxane copolymers, dimethylpolysiloxanes containing dimethylvinylsiloxy end groups, dimethylsiloxane-methylphenylsiloxane copolymers containing dimethylvinylsiloxy end groups, dimethylsiloxane-diphenylsiloxane methylvinylsiloxane copolymers containing dimethylvinylsiloxy end groups, dimethylsiloxane-methylvinylsiloxane copolymers containing trimethylsiloxy end groups, dimethylsiloxane-methylphenylsiloxane-methylvinylsiloxane copolymers containing trimethylsiloxy end groups, methyl (3,3,3-trifluoropropyl)-polysiloxane containing dimethylvinylsiloxy end groups, and dimethylsiloxanemethyl (3,3,3-trifluoropropyl)siloxane copolymers containing dimethylvinylsiloxy end groups.

In particular, the organopolysiloxane elastomer can be obtained by reaction of dimethylpolysiloxane containing dimethylvinylsiloxy end groups and methyl hydrogeno-polysiloxane containing trimethylsiloxy end groups, in the presence of a platinum catalyst.

Advantageously, the sum of the number of ethylenic groups per molecule of the compound (B) and the number of hydrogen atoms bonded to silicon atoms per molecule of the compound (A) is at least 5.

It is advantageous for the compound (A) to be added in an amount such that the molar ratio between the total amount of hydrogen atoms bonded to silicon atoms in the compound (A) and the total amount of all such ethylenically unsaturated groups in compound (B) is in the range of 1.5/1 to 20/1.

The compound (C) is the catalyst for the crosslinking reaction, and 25 is in particular chloroplatinic acid, the chloroplatinic acid-olefin complexes, chloroplatinic acid-alkenylsiloxane complexes, the chloroplatinic acid-diacetone complexes, platinum black, and platinum on a support. The catalyst (C) is preferably added in a proportion of 0.1 to 1000 parts by weight, preferably 1 to 100 parts by weight, by way of metal platinum proper added in a proportion of 1,000 parts by weight of the total amount of compounds (A) and B). The elastomer is advantageously a non-emulsifying elastomer.

The term “non-emulsifying” is understood to define organopolysiloxane elastomers not containing any hydrophilic chain, and in particular containing no polyoxyalkylene units (in particular polyoxyethylene or polyoxypropylene), nor any polyglyceryl unit. Thus, according to one particular embodiment of the invention, the composition comprises an organopolysiloxane elastomer free of both polyoxyalkylene units and polyglyceryl units. In particular, the silicone elastomer used in the present invention is selected from among Dimethicone Crosspolymer (INCI name), Vinyl Dimethicone Crosspolymer (INCI name), Dimethicone/Vinyl Dimethicone Crosspolymer (INCI name), Dimethicone Crosspolymer-3 (INCI name). The organopolysiloxane elastomer particles can be conveyed in the form of a gel constituted of an elastomeric organopolysiloxane included in at least one hydrocarbon oil and/or one silicone oil. In these gels, the organopolysiloxane particles are often non-spherical particles.

Non-emulsifying elastomers are in particular described in the patents EP 242 219, EP 285 886, EP 765 656 and in the patent application JP-A-61-194009.

The silicone elastomer is generally present in the form of a gel, a paste or a powder but preferably advantageously in the form of a gel in which the silicone elastomer is dispersed in a linear silicone oil (dimethicone) or cyclic silicone oil (eg cyclopentasiloxane), advantageously in a linear silicone oil.

By way of non-emulsifying elastomers, use may be made particularly of those marketed under the trade names “KSG-6”, “KSG-15”, “KSG-16”, “KSG-18”, “KSG-41”, “KSG-42” “KSG-43”, “KSG-44” by the company Shin Etsu, “DC9040”, “DC9041” by the company Dow Corning, “SFE 839” by the company General Electric.

According to one particular embodiment, use is made of a silicone elastomer gel dispersed in a silicone oil selected from a non-exhaustive list comprising cyclopentadimethylsiloxane, dimethicones, dimethylsiloxane, methyl trimethicone, phenylmethicone, phenyl dimethicone, phenyl trimethicone, and cyclomethicone, preferably a linear silicone oil selected from the polydimethylsiloxanes (PDMS), or dimethicones having a viscosity at 25° C. ranging from 1 to 500 cst at 25° C., optionally modified by aliphatic groups, optionally fluorinated 3025096 46, or by functional groups such as hydroxyl groups, thiols and/or amines.

Mention may be made in particular of the following compounds listed by their INCI names:
Dimethicone/Vinyl Dimethicone Crosspolymer, such as “USG-105” and “USG-107A” from the company Shin-Etsu; “DC9506” and “DC9701” from the company Dow Corning;
Dimethicone/Vinyl Dimethicone Crosspolymer (and) Dimethicone, such as “KSG-6” and “KSG-16” from the company Shin Etsu;
Dimethicone/Vinyl Dimethicone Crosspolymer (and) Cyclopentasiloxane, such as “KSG-15”;
Cyclopentasiloxane (and) Dimethicone Crosspolymer, such as “DC9040”, “DC9045” and “DC5930” from the company Dow Corning;
Dimethicone (and) Dimethicone Crosspolymer, such as “DC9041” from the company Dow Corning;
Dimethicone (and) Dimethicone Crosspolymer, such as “Dow Corning EL-9240® silicone elastomer blend” from the company Dow Corning (mixture of polydimethylsiloxane crosslinked with hexadiene/polydimethylsiloxane (2 cSt));
C₄₋₂₄ Alkyl Dimethicone/DivinylDimethicone Crosspolymer, such as NuLastic Silk MA by the company Alzo.

By way of examples of silicone elastomers dispersed in a linear silicone oil that may advantageously be used according to the invention, mention may be made notably of the following references:
Dimethicone/Vinyl Dimethicone Crosspolymer (and) Dimethicone, such as “KSG-6” and “KSG-16” from the company Shin Etsu;
Dimethicone (and) Dimethicone Crosspolymer, such as “DC9041” from the company Dow Corning;
Dimethicone (and) Dimethicone Crosspolymer, such as “Dow Corning EL-9240® silicone elastomer blend” from the company Dow Corning (mixture of polydimethylsiloxane crosslinked by Hexadiene/Polydimethylsiloxane (2 cSt)); and
DIMETHICONE (and) VINYLDIMETHYL/TRIMETHYLSILOXYSILICATE/DIMETHICONE CROSSPOLYMER, BELSIL REG 1100 from Wacker Silicone.

The particles of organopolysiloxane elastomers may also be used in powder form, mention may be made of the powders marketed under the trade name “Dow Corning 9505 Powder”, “Dow Corning 9506 Powder” from the company Dow Corning, these powders are known under the INCI name: dimethicone/vinyl dimethicone crosspolymer, as well as “Dow Corning® 9701 Cosmetic Powder” (INCI: Dimethicone/Vinyl dimethicone Crosspolymer (and) Silica).

The organopolysiloxane powder may also be coated with silsesquioxane resin, as described for example in the U.S. Pat. No. 5,538,793. Such elastomeric powders are marketed under the trade names “KSP-100”, “KSP-101”, “KSP-102”, “KSP-103”, “KSP-104”, “KSP-105” by the company Shin Etsu, and are also known by their INCI name: vinyl dimethicone/methicone silsesquioxane crosspolymer.

By way of examples of organopolysiloxane powders coated with silsesquioxane resin that may be used advantageously according to the invention, mention may be made notably of the reference “KSP-100” from the company Shin Etsu.

By way of a preferred lipophilic gelling agent of the organopolysiloxane elastomer type, in particular mention may be made of cross-linked organopolysiloxane elastomers selected from among Dimethicone Crosspolymer (INCI name), Dimethicone (and) Dimethicone Crosspolymer (INCI name), Vinyl Dimethicone Crosspolymer (INCI name), Dimethicone/Vinyl Dimethicone Crosspolymer (INCI name), Dimethicone Crosspolymer-3 (INCI name), DIMETHICONE (and) VINYLDIMETHYL/TRIMETHYLSILOXYSILICATE/DIMETHICONE CROSSPOLYMER and in particular DIMETHICONE (and) DIMETHICONE/VINYL DIMETHICONE CROSSPOLYMER, KSG16 from the company Shin Etsu, or indeed DIMETHICONE (and) VINYLDIMETHYL/TRIMETHYLSILOXYSILICATE/DIMETHICONE CROSSPOLYMER, BELSIL REG 1100 KSG16 from the company Wacker Silicone.

Semi-Crystalline Polymers

The composition according to the invention may comprise at least one semi-crystalline polymer. Preferably, the semi-crystalline polymer has an organic structure, and a melting point temperature greater than or equal to 30° C.

The term “semi-crystalline polymer” within the meaning of the invention is understood to refer to polymers comprising a crystallisable moiety and an amorphous moiety and having a reversible phase change temperature of the first order, in particular of melting (solid-liquid transition). The crystallisable moiety is either a side chain (or pendent chain), or a block in the backbone. When the crystallisable moiety of the semi-crystalline polymer is a block of the polymer backbone, this crystallisable block has a chemical nature that is different from that of the amorphous blocks; the semi-crystalline polymer is in this case a block copolymer for example of the diblock, triblock or multiblock type. When the crystallisable moiety is a pendant chain to the backbone, the semi-crystalline polymer may be a homopolymer or a copolymer. The melting temperature of the semi-crystalline polymer is preferably less than 150° C. The melting temperature of the semi-crystalline polymer is preferably greater than or equal to 30° C. and less than 100° C. More preferably, the melting temperature of the semi-crystalline polymer is greater than or equal to 30° C. and lower than 70° C. The one or more semi-crystalline polymer(s) according to the invention are solid at ambient temperature (25° C.) and atmospheric pressure (760 mm Hg), and have a melting point temperature that is greater than or equal to 30° C. The melting point values correspond to the melting point measured by using a differential scanning calorimeter (DSC), such as the calorimeter marketed under the trade name DSC 30 by the company Mettler, with a rise in temperature of 5 or 10° C. per minute (the melting point considered is the point corresponding to the temperature of the most endothermic peak of the thermogram).

The one or more semi-crystalline polymer(s) according to the invention preferably have a melting temperature higher than the temperature of the keratinous substrate intended for receiving the said composition, in particular the skin, the lips or the eyelids.

According to the invention the semi-crystalline polymers are advantageously soluble in the fatty phase, in particular to at least 1% by weight, at a temperature above their melting temperature. Apart from the crystallisable chains or blocks, the polymer blocks are amorphous. The term “crystallisable chain or block” within the meaning of the invention is understood to refer to a chain or block which if it were alone would change states passing from the amorphous to the crystalline state in a reversible manner, depending on whether it is above or below the melting point temperature. A chain within the meaning of the invention is a group of atoms, being a pendant or side chain relative to the backbone of the polymer. A block is a group of atoms belonging to the backbone, a group constituting one of the repeating units of the polymer.

Preferably, the polymer backbone of the semi-crystalline polymers is soluble in the fatty phase at a temperature above their melting temperature. Preferably, the crystallisable blocks or chains of the semi-crystalline polymers represent at least 30% of the total weight of each polymer and more preferably at least 40%. The semi-crystalline polymers containing crystallisable side chains are homo- or copolymers. The semi-crystalline polymers of the invention containing crystallisable blocks are copolymers, blocks or multiblocks. They can be obtained by polymerisation of reactive double bond (or ethylenic) monomer or by polycondensation. When the polymers of the invention are polymers containing crystallisable side chains, the latter are advantageously in random or statistical form.

Preferably, the semi-crystalline polymers of the invention are of synthetic origin.

According to one preferred embodiment, the semi-crystalline polymer is selected from among:
homopolymers and copolymers comprising units resulting from the polymerisation of one or more monomers bearing crystallisable hydrophobic side chain(s);
the polymers that bear in the backbone at least one crystallisable block;
polycondensates of polyester, aliphatic, or aromatic, or aliphatic/aromatic type;
copolymers of ethylene and propylene prepared by metallocene catalysis; and
acrylate/silicone copolymers.

The semi-crystalline polymers that may be used in the invention may be selected in particular from among:
block copolymers of polyolefins with controlled crystallisation, whereof the monomers are described in the patent EP 0951897;
polycondensates, and in particular of the polyester, aliphatic, or aromatic, or aliphatic/aromatic type;
copolymers of ethylene and propylene prepared by metallocene catalysis;
homopolymers or copolymers bearing at least one crystallisable side chain and homopolymers or copolymers bearing in the backbone at least one crystallisable block, such as those described in the document U.S. Pat. No. 5,156,911, such as (C₁₀-C₃₀)alkyl polyacrylates corresponding to Intelimer® from the company Landec described in the brochure “Intelimere polymers”, Landec 1 P22 (Rev. 4-97) and for example the product Intelimer® IPA 13-1 from the company Landec, which is a stearyl polyacrylate having a molecular weight of about 145 000 and whose melting temperature is equal to 49° C.;
homopolymers or copolymers bearing at least one crystallisable side chain, in particular with fluorinated group(s), as described in the patent document WO 01/19333;
acrylates/silicone copolymers, such as acrylic acid and stearyl acrylate-polydimethylsiloxane graft copolymers, stearyl methacrylate-polydimethylsiloxane graft copolymer, acrylic acid and stearyl methacrylate-polydimethylsiloxane graft copolymers, methyl methacrylate copolymers, butyl methacrylate, ethyl acrylate 2-hexyl and stearyl methacrylate-polydimethylsiloxane grafts. Mention may be made in particular of the copolymers marketed by the company SHIN-ETSU under the trade names KP-561 (CTFA name: acrylates/dimethicone), KP-541 (CTFA name: acrylates/dimethicone and isopropyl alcohol), KP-545 (CTFA name: acrylates/dimethicone and Cyclopentasiloxane);
and the mixtures thereof.

Polyacrylates

According to one embodiment, the gelling agent is selected from polyacrylates resulting from the polymerisation of C₁₀-C₃₀ alkyl acrylate(s), preferably C₁₄-C₂₄ alkyl acrylate(s), and even more preferentially C₁₈-C₂₂ alkyl acrylate(s).

According to one embodiment, the polyacrylates are polymers of acrylic acid esterified with a fatty alcohol whose saturated carbon chain comprises from 10 to 30 carbon atoms, preferably from 14 to 24 carbon atoms, or a mixture of the said fatty alcohols. Preferably, the fatty alcohol comprises 18 carbon atoms or 22 carbon atoms.

Among the polyacrylates, mention may be made more particularly of stearyl polyacrylate, behenyl polyacrylate. Preferably, the gelling agent is stearyl polyacrylate, or behenyl polyacrylate.

Mention may be made in particular of polyacrylates marketed under the trade names Interlimer® (INCI name: Poly C₁₀-C₃₀ alkyl acrylate), in particular Interlimer® 13.1 and Interlimer® 13.6, by the company Airproducts.

Dextrin Esters

The composition according to the invention may comprise at least one dextrin ester. In particular, the composition preferably comprises at least one ester of dextrin and of fatty acid, preferably having C₁₂ to C₂₄, in particular C₁₄ to C₁₈, or the mixtures thereof. Preferably, the dextrin ester is an ester of dextrin and of fatty acid having C₁₂-C₁₈, in particular C₁₄-C₁₈.

Preferably, the dextrin ester is selected from dextrin myristate and/or dextrin palmitate, and the mixtures thereof.

According to one particular embodiment, the dextrin ester is dextrin myristate, such as that marketed in particular under the trade name Rheopearl MKL-2 by the company Chiba Flour Milling.

According to one preferred embodiment, the dextrin ester is dextrin palmitate. The latter may for example be selected from those marketed under the trade names Rheopearl TL® or Rheopearl KL® or Rheopearl® KL2 by the company Chiba Flour Milling.

Inulin Ester

The composition according to the invention may comprise at least one ester of Inulin and fatty acid. Mention may be made in particular of esters of inulin and fatty acid(s) marketed under the trade names Rheopearl® ISK2 or Rheopearl® ISL2 (INCI name: Stearoyl Inulin) by the company Miyoshi Europe

Glycerol Ester

The composition according to the invention may comprise at least one ester of glycerol and fatty acid(s), in particular a mono-, di- or triester of glycerol and fatty acid(s). Typically the said ester of glycerol and fatty acid(s) may be used alone or in admixture.

According to the invention, it may be an ester of glycerol and a fatty acid or an ester of glycerol and a fatty acid mixture.

According to one embodiment, the fatty acid is selected from the group constituted of behenic acid, isooctadecanoic acid, stearic acid, arachidic acid, and the mixtures thereof.

Mention may be made in particular of the esters of glycerol and of fatty acid(s) marketed under the trade names Nomcort HK-G (INCI name: Glyceryl behenate/eicosadioate) and Nomcort SG (INCI name: Glyceryl tribehenate, isostearate, eicosadioate) by the company Nisshin Oillio.

Hydrogen Bonding Polymers

As representative examples of hydrogen bonding polymers suitable for the invention, mention may be made particularly of polyamides, and in particular hydrocarbon polyamides and silicone polyamides.

The fatty phase of a composition according to the invention may comprise at least one polyamide selected from among hydrocarbon polyamides, silicone polyamides, and the mixtures thereof. The term “polyamide” within the meaning of the invention is understood to refer to a compound having at least two amide repeating units, preferably at least three amide repeating units and even more preferably 10 amide repeating units.

The term “hydrocarbon-based polyamide” is understood to refer to a polyamide essentially formed, or indeed even constituted, of carbon and hydrogen atoms, and optionally oxygen atoms, nitrogen atoms, and containing no silicon or fluorine atoms. It may comprise alcohol, ester, ether, carboxylic acid, amine and/or amide. The term “functionalised chain” within the meaning of the invention is understood to refer to an alkyl chain comprising one or more functional or reactive groups in particular selected from among hydroxyl, ether, esters, oxyalkylene or polyoxyalkylene. Advantageously, this polyamide of the composition according to the invention has an average molecular weight of less than 100,000 g/mol, in particular ranging from 1000 to 100,000 g/mol, in particular less than 50,000 g/mol, in particular ranging from 1000 to 50,000 g/mol, more particularly ranging from 1000 to 30,000 g/mol, preferably from 2000 to 20,000 g/mol, and better still from 2000 to 10,000 g/mol. This polyamide is not soluble in water, particularly at 25° C.

According to a first embodiment of the invention the polyamide used is a polyamide having the formula (I):

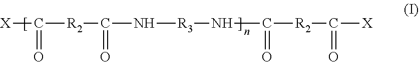

wherein X represents a group —N(R₁)₂ or a group —OR₁ wherein R₁ is a linear or branched, C₈ to C₂₂ alkyl radical, which may be identical or different from each other, R₂ is a residue of C₂₈-C₄₂ dimer diacid, R₃ is an ethylene diamine radical, n is comprised between 2 and 5; and the mixtures thereof.

According to one particular embodiment, the polyamide used is a polyamide with amide end groups having the formula (Ia):

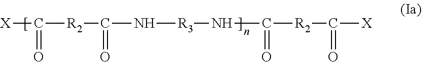

wherein X represents a group —N(R₁)₂ wherein R₁ is a linear or branched, C₈ to C₂₂ alkyl radical, which may be identical or different from each other, R₂ is a residue of C₂₈-C₄₂ dimer diacid, R₃ is an ethylene diamine radical, n is comprised between 2 and 5; and the mixtures thereof.

The fatty phase of a composition according to the invention may further comprise, in an additional manner in this case, at least one additional polyamide having the formula (Ib):

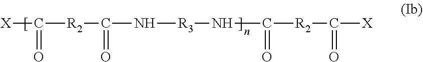

wherein X represents a group —OR₁ wherein R₁ is a linear or branched, C₈ to C₂₂ alkyl radical, which may be identical or different from each other, R₂ is a residue of C₂₈-C₄₂ dimer diacid, R₃ is an ethylene diamine radical, n is comprised between 2 and 5; such as the commercial products marketed by the company Arizona Chemical under the trade names Uniclear 80 and Uniclear 100 or even Uniclear® 80 V, Uniclear® 100 V and Uniclear® 100 VG, which correspond to the INCI name “ethylenediamine/stearyl dimer dilinoleate copolymer.”

The silicone polyamides are preferably solid at ambient temperature (25° C.) and atmospheric pressure (760 mm Hg). The silicone polyamides may preferably be polymers comprising at least one unit having the formula (III) or (IV):

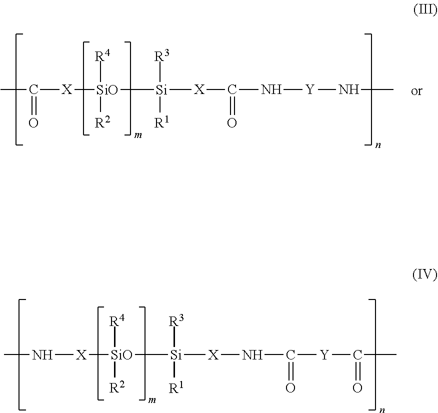

in which:
R⁴, R⁵, R⁶ and R⁷, which may be identical or different, represent a group selected from among:
saturated or unsaturated hydrocarbon groups, being linear, branched or cyclic, having C₁ to C₄₀, which may possibly contain in their chain one or more atoms of oxygen, sulfur and/or nitrogen, and may be substituted in part or entirely by fluorine atoms;
aryl groups, having C₆ to C₁₀, possibly substituted by one or more alkyl groups having C₁ à C₄;
polyorganosiloxane chains whether or not containing one or more atoms of oxygen, sulfur and/or nitrogen;
the X, which may be identical or different, represent a linear or branched, alkylene di-yl group, having C₁ to C₃₀, which may possibly contain in their chain one or more atoms of oxygen and/or nitrogen,
Y is a linear or branched divalent alkylene, arylene, cycloalkylene, alkylarylene or arylalkylene group, saturated or unsaturated, having C₁ to C₅₀, which may comprise one or more atoms of oxygen, sulfur and/or nitrogen, and/or bear as substituent one of the following atoms or groups of atoms: fluorine, hydroxy, C₃ to C₈ cycloalkyl, C₁ to C₄₀ alkyl, C₅ to C₁₀ aryl, phenyl possibly substituted by 1 to 3 groups of C₁ to C₃ alkyl, C₁ to C₃ hydroxyalkyl and C₁ to C₆ amino alkyl, or

Y represents a group having the formula

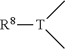

in which
T represents a saturated or unsaturated, linear or branched, trivalent or tetravalent hydrocarbon group, having C₃-C₂₄ possibly substituted by a polyorganosiloxane chain, and possibly containing one or more atoms selected from O, N and S, or T represents a trivalent atom selected from N, P and Al, and
R⁸ represents a linear or branched, C₁ to C₅₀ alkyl group, or a polyorganosiloxane chain, that may comprise one or more ester, amide, urethane, thiocarbamate, urea, thiourea and/or sulfonamide groups which may or may not be bound to another chain of the polymer,
n is an integer in a range from 2 to 500, preferably from 2 to 200 and m is an integer in a range from 1 to 1000, preferably from 1 to 700 and better still from 6 to 200.

According to one particular embodiment, the silicone polyamide comprises at least one unit having the formula (III) wherein m is in a range from 50 to 200, in particular from 75 to 150, and preferably is of the order of 100.

More preferably R⁴, R⁵, R⁶ and R⁷ independently represent a linear or branched, C₁-C₄₀ alkyl group, preferably a CH₃, C₂H₅, n-C₃H₇ or isopropyl group in the formula (III).

By way of examples of silicone polymer that may be used mention may be made of one of the silicone polyamides obtained in accordance with Examples 1 to 3 of document U.S. Pat. No. 5,981,680. Mention may be made of the compounds marketed by the company Dow Corning under the trade names DC 2-8179 (DP 100) and DC 2-8178 (DP 15), corresponding to the INCI name “Nylon611/dimethicone copolymers” that is to say copolymers of Nylon-611/dimethicone.

The polymers and/or silicone copolymers advantageously have a transition temperature when transitioning from the solid state to the liquid state ranging from 45° C. to 190° C. Preferably, they present a temperature of transition from the solid state to the liquid state ranging from 70° C. to 130° C. and preferably 80° C. to 105° C.

Hydrocarbon Block Copolymer

Hydrocarbon block copolymers, also referred to as block copolymers are selected from those that are able to thicken or gel the fatty phase of the composition.

The term “amorphous polymer”, is understood to refer to a polymer that has no crystalline form. The polymeric gelling agent is also preferably film-forming, that is-to-say, it is capable of forming a film when applied on the skin and/or lips.

The hydrocarbon block copolymer can in particular be a diblock, triblock, multiblock, radial, or star copolymer, or the mixtures thereof. Such hydrocarbon block copolymers are described in the patent application US-A-2002/005 562 and in the U.S. Pat. No. 5,221,534. The copolymer may have at least one block whose glass transition temperature is preferably less than 20° C., preferably less than or equal to 0° C., preferably less than or equal to −20° C., more preferably less than or equal to −40° C. The glass transition temperature of the said block may be comprised between −150° C. and 20° C., in particular between −100° C. and 0° C. The hydrocarbon block copolymer present in the composition according to the invention is an amorphous copolymer formed by polymerisation of an olefin. The olefin may be in particular an elastomeric ethylenically unsaturated monomer. By way of examples of olefin, mention may be made of ethylene carbide monomers [unsaturated hydrocarbon monomers] having in particular one or two ethylenic unsaturations, having from 2 to 5 carbon atoms such as ethylene, propylene, butadiene, isoprene or pentadiene.

Advantageously, the hydrocarbon-based block copolymer is an amorphous block copolymer of styrene and olefin.

In particular it is preferred for block copolymers to include at least one styrene block and at least one block comprising units selected from butadiene, ethylene, propylene, butylene, isoprene or a mixture thereof.

According to one preferred embodiment, the hydrocarbon-based block copolymer is hydrogenated to reduce the residual ethylenic unsaturations after polymerisation of the monomers. In particular, the hydrocarbon-based block copolymer is an optionally hydrogenated copolymer comprising styrene blocks and ethylene/C3-C4 alkylene blocks.

According to one preferred embodiment, the composition according to the Invention comprises at least one diblock copolymer, preferably hydrogenated, preferably selected from among styrene-ethylene/propylene copolymers, styrene-ethylene/butadiene copolymers, styrene-ethylene/butylene copolymers. Diblock polymers are in particular marketed under the trade name Kraton G1701 E by the company Kraton Polymers. Advantageously, use as a polymeric gelling agent is made of a diblock copolymer such as those described here above, in particular a styrene-ethylene/propylene diblock copolymer, or a diblock mixture, as previously described here above.

Thus, according to one embodiment, a composition according to the invention comprises, as lipophilic gelling agent at least one hydrocarbon block copolymer, preferably an optionally hydrogenated copolymer comprising styrene blocks and ethylene/C3-C4 alkylene blocks, still more preferentially selected from a diblock copolymer, preferably hydrogenated, such as a styrene-ethylene/propylene copolymer, a styrene-ethylene/butadiene copolymer.

By way of a lipophilic agent having suspending capacity, mention can also be made of EstoGel M (INCI: CASTOR OIL/IPDI COPOLYMER & CAPRYLIC/CAPRIC TRIGLYCERIDES) marketed by the company Polymer Expert.

The person skilled in the art will be careful in selecting the nature and/or quantity of lipophilic agent(s) having suspending capacity in a manner so as to achieve a desired level of viscosity of the fatty phase, and in particular to achieve the desired suspension character (or power) for suspending the said desired fatty phase, in particular vis-à-vis the spheres (S1). These adjustments fall within the domain of general knowledge of the person skilled in the art.

According to one embodiment, in the compositions of the invention, the content of lipophilic agent(s) having suspending capacity is comprised between 0.5% and 99.50%, preferably between 1.5% and 70%, in particular between 2.5% and 60%, and preferentially between 3% and 50%, or indeed even between 1% and 8%, and more preferably between 2.5% and 6% by weight in relation to the total weight of the fatty phase, or indeed relative to the total weight of the composition.

Oils

The fatty phase of a composition according to the invention may further comprise at least one oil.

The fatty phase may therefore comprise one single oil or a blend of several oils. The fatty phase according to the invention may thus comprise at least one, at least two, at least three, at least four, at least five, or indeed more, oil(s) as described here below.

The term “oil” is understood to refer to a fatty substance that is liquid at ambient temperature (25° C.).

By way of oils that may be used in the composition according to the invention mention may be made for example of:
hydrocarbon oils of plant origin, as described here below;
hydrocarbon oils of animal origin, such as perhydrosqualene and squalane;
esters and synthetic ethers, in particular of fatty acids, such as oils having the formulas R₁COOR₂ and R₁OR₂ wherein R represents the residue of a C₈-C₂₉ fatty acid and R₂ represents a branched or unbranched, C₃-C₃₀, hydrocarbon chain, such as Purcellin oil, isononyl isononanoate, isodecyl neopentanoate, isopropyl myristate, ethyl-2-hexyl palmitate, octyl-2-dodecyl stearate, octyl-2-dodecyl erucate, isostearyl isostearate; hydroxylated esters such as isostearyl lactate, octylhydroxystearate, octyldodecyl hydroxystearate, diisostearyl malate, triisocetyl citrate, heptanoates, octanoates, decanoates of fatty alcohols; polyol esters, such as propylene glycol dioctanoate, neopentyl glycol diheptanoate, and diethylene glycol diisononanoate; and pentaerythritol esters such as pentaerythrityl tetrabehenate (DUB PTB) or pentaerythrityl tetraisostearate (Prisorine 3631), or Plandool G (INCI: Bis-Behenyl/Isostearyl/Phytosteryl/Dimer Dilinoleyl Dimer Dilinoleate);
linear or branched hydrocarbons, of mineral or synthetic origin, such as volatile or nonvolatile paraffin oils, and derivatives thereof, petroleum jelly, polydecenes, hydrogenated polyisobutene such as Parleam oil;
silicone oils, such as for example volatile or non-volatile, polydimethylsiloxanes (PDMS) with linear or cyclic silicone chain, which are liquid or pasty at ambient temperature, in particular cyclopolydimethylsiloxanes (cyclomethicones) such as cyclohexasiloxane and cyclopentasiloxane; polydimethylsiloxanes (or dimethicones) comprising alkyl, alkoxy or phenyl groups, pendent or at the end of a silicone chain, these groups containing from 2 to 24 carbon atoms; phenyl silicones such as phenyltrimethicones, phenyldimethicones, phenyltrimethylsiloxydiphenyl-siloxanes, diphenyl-dimethicones, diphenylmethyldiphenyl trisiloxanes, 2-phenylethyltrimethylsiloxysilicates, and polymethylphenylsiloxanes;
fatty alcohols containing from 8 to 26 carbon atoms, such as cetyl alcohol, stearyl alcohol and blend thereof (cetylstearyl alcohol), or even octyldodecanol;
partially hydrocarbon-based and/or silicone-based fluoro oils such as those described in the document JP-A-2 295 5912;
and the mixtures thereof.

According to one preferred embodiment, the oil is selected from synthetic ethers and esters, preferably esters having the formula R₁COOR₂, wherein R represents a fatty acid residue containing from 8 to 29 carbon atoms (C₈-C₂₉), and R² represents a branched or unbranched hydrocarbon-based chain containing from 3 to 30 carbon atoms (C₃-C₃₀).

According to one embodiment, the oil is selected from fatty alcohols having from 8 to 26 carbon atoms.

According to one embodiment, the oil is selected from among hydrocarbon-based oils having from 8 to 16 carbon atoms, and in particular branched alkanes having from 8 to 16 carbon atoms (C₈-C₁₆) (also known as isoparaffins or isoalkanes), such as isododecane (also known as 2-methylundecane) isodecane, isohexadecane and, for example, the oils marketed under the trade names Isopar® or Permethyls®.

According to another preferred embodiment, the fatty phase of the compositions of the invention comprises an oil selected from among silicone oils. Preferably, the fatty phase does not comprise other oils different from silicone oils. Preferably the oils contained in the fatty phase are silicone oils.

According to one preferred embodiment, a composition according to the invention comprises at least 1% by weight of oil(s) relative to the total weight of the said composition.

According to one other embodiment, a composition according to the invention, in particular the fatty phase of the compositions of the invention, does not comprise polydimethylsiloxane (PDMS), and preferably does not comprise a silicone oil.

According to one other embodiment, a composition according to the invention does not comprise a plant oil.

In yet another embodiment, the fatty phase of the compositions according to the invention comprises at least one hydrocarbon oil of plant origin. By way of plant oils, particular mention can be made of liquid triglycerides of C₄-C₁₀ fatty acids, such as the triglycerides of heptanoic or octanolc acids, or for example sunflower seed, corn, soybean, pumpkin, grapeseed, sesame, hazelnut, apricot, macadamia arara, castor, and avocado oils, the triglycerides of caprylic/capric acids (INCI name: Caprylic/Capric Triglyceride) such as those marketed by the company Stearineries Dubois or those available under the trade names “Miglyol 810”, “Miglyol 812” and “Miglyol 818 by the company Dynamit Nobel, jojoba oil, Shea butter oil, and the mixtures thereof.

Preferably, the plant oil is selected from among those high in polyunsaturated fatty acids. The term “unsaturated fatty acid” within the meaning of the present invention, is understood to refer to a fatty acid comprising at least one double bond. They are most particularly long-chain fatty acids, that is to say having more than 14 carbon atoms. The unsaturated fatty acids can be in acid form or salt form such as for example their calcium salt, or even in the form of derivatives in particular esters(s) of fatty acid(s).

Preferably the continuous fatty phase comprises at least one non-volatile oil.

The term “non-volatile” is understood to refer to an oil having a vapour pressure at ambient temperature and atmospheric pressure that is non-zero and lower than 0.02 mm Hg (2.66 Pa) and better still lower than 10⁻³ mm Hg (0.13 Pa).

In particular, mention may be made of non-volatile oils selected from among silicone oils, fluorinated oils or the mixtures thereof, and more particularly from non-volatile, non-phenylated silicone oils; the non-volatile, non-phenylated silicone oils, whether or not having at least one dimethicone fragment; fluorinated oils; or the mixtures thereof, or even polar, non-volatile hydrocarbon-based oils, in particular selected from among non-volatile oils comprising either none or at the very most only one free hydroxyl group, or from non-volatile oils comprising at least two free hydroxyl groups, or from among non-polar, non-volatile, hydrocarbon-based oils.

Representative examples of non-volatile, non-phenylated silicone oils that may be mentioned include polydimethylsiloxanes; alkyldimethicones; vinylmethylmethicones; and also silicones modified with aliphatic groups and/or with functional groups such as hydroxyl groups, thiols and/or amines.

Among the polar non-volatile hydrocarbon-based oils, mention may be made of ester oils as described here above.

Among the apolar, non-volatile hydrocarbon-based oils, mention may be made of linear or branched hydrocarbons, of mineral or synthetic origin such as:
paraffin oil or its derivatives,
squalane,
isoeicosane,
naphthalene oil,
polybutylenes such as INDOPOL H-100 (molar mass or MW=965 g/mol), INDOPOL H-300 (MW=1340 g/mol), INDOPOL 141500 (MW=2160 g/mol) marketed or manufactured by the company AMOCO,
polyisobutenes
hydrogenated polyisobutylenes such as Parleam® marketed by the company NIPPON OIL FATS, PANALANE H-300 E marketed or manufactured by AMOCO (MW=1340 g/mol), VISEAL 20000 marketed or manufactured by the SYNTEAL company (MW=6000 g/mol), REWOPAL PIB 1000 marketed or manufactured by the company WITCO (MW=1000 g/mol), or even PARLEAM LITE marketed by the company NOF Corporation,
decene/butene copolymers, polybutene/polyisobutene copolymers in particular Indopol L-14,
polydecenes and hydrogenated polydecenes such as: PURESYN 10 (MW=723 g/mol), PURESYN 150 (MW=9200 g/mol) marketed or manufactured by the company MOBIL CHEMICALS, or indeed even PURESYN 6 marketed by the company EXXONMOBIL CHEMICAL)
and the mixtures thereof.

According to one preferred embodiment, the oil is selected from the group consisting of isononyl isononanoate, dimethicone, polybutene, whether or not hydrogenated, diisostearyl malate, and the mixtures thereof.

The selection of oil(s) that satisfy the criterion of glossy lustre desired for a composition according to the invention falls within domain of general knowledge of the person skilled in the art.

According to one embodiment, the content of oil(s) is comprised between 0.5% and 99% by weight in relation to the total weight of the fatty phase of the said composition. Preferably, the content of oil(s) is greater than 70%, in particular greater than 80%, or Indeed even greater than 90%, by weight in relation to the weight of the fatty phase.

Regardless of the colouring agent(s) or dye(s) used in implementation (as mentioned farther below), the fatty phase of the composition according to the invention remains perfectly transparent, this transparency not being attainable with conventional glosses/lipsticks in the form of inverse emulsions. It is also an advantage compared to an emulsion with an anionic polymer coacervate (carbomer)/cationic polymer (amodimethicone) obtained via a microfluidic method, as described in particular in the application WO 2012/120043, where amodimethicone tends to cause “cloudiness” in the fatty phase.

Additional Compounds

According to the invention, the aqueous phase and/or the fatty phase may in addition comprise at least one additional compound that is different from the hydrophilic gelling agents, lipophilic agents having suspending capacity and oils, or indeed the anionic and cationic polymers, mentioned here above.

A composition according to the invention, in particular the aqueous phase and/or the fatty phase of the said composition, may thus further comprise by way of an additional compound powders, flakes, colouring agents, in particular selected from among colouring agents, whether or not water-soluble, whether or not liposoluble, organic or inorganic, from pigments, optical effect providing materials, liquid crystals, and mixtures thereof, particulate agents that are insoluble in the fatty phase, emulsifying and/or non emulsifying silicone elastomers in particular as described in the patent application EP 2 353 577, preservatives, humectants, stabilisers, chelating agents, emollients, modifying agents, selected from agents acting on pH, and on osmotic force, and/or refraction index modifiers etc. . . . or any usual cosmetic additives, and the mixtures thereof.

A composition according to the invention, in particular the aqueous phase and/or the fatty phase of the said composition, may indeed in addition include at least one active agent, in particular a biological or cosmetic substance, preferably selected from among moisturising/hydrating agents, healing agents, depigmenting agents, UV filters, desquamating agents, antioxidants, active substances that stimulate dermal and/or epidermal macromolecular synthesis, dermodecontracting agents, antiperspirant agents, soothing agents, anti-aging agents, perfuming agents and the mixtures thereof. Such active agents are described in the patent application FR 1558849.

In particular, the fatty phase may also comprise at least one hydrophobic film-forming polymer, in particular as described in the patent applications FR 3025100 or WO 2016/030842, and for example the polymer marketed under the trade references FA 4002 ID (TIB 4-202) or FA 4001 CM (TIB 4-230) by the company Dow Corning. The presence of such a polymer serves to improve the performance and staying power of the product over time, in particular the durability over an extended period of the glossy lustre, and where necessary while also maintaining the fatty phase at a desirable level of viscosity compatible with the microfluidic device. In addition, it makes it possible to reduce the migration phenomena of the composition applied over a keratin material, particularly the skin or eye lids.

According to one embodiment, the content by weight of the hydrophobic film-forming polymer(s) is comprised between 0.1% and 40%, in particular between 0.2% and 20%, preferably between 0.5% and 15%, in relation to the weight of the fatty phase.

According to one particular embodiment, a composition according to the invention does not include a hydrophilic film-forming polymer, in particular as described in the patent document FR 3025100, and/or tackifying resin, such as described in FR 3025099.

Obviously, the person skilled in the art will exercise care in selecting the possible optional additional compound(s) and/or active agent(s) mentioned above and/or the respective quantities thereof in a manner so as to ensure that the advantageous properties of the composition according to the invention are either not at all or not substantially impaired by the envisaged addition. In particular, the nature and/or quantity of the additional compound(s) and/or active agent(s) are dependent on the aqueous or fatty nature of the particular phase considered of the composition according to the invention. These adjustments fall within the expertise and general knowledge of the person skilled in the art.

Colouring Agent

According to one embodiment, a composition according to the invention comprises at least one colouring agent.

According to one embodiment, the aqueous phase and/or the fatty phase comprises/comprise at least one colouring agent.

When the fatty phase of a composition according to the invention comprises at least one colouring agent, the said composition exhibits an advantageous compromise between “transparency/lip colouring”. Indeed, in the presence of such a colouring agent, although the dispersion is coloured it nevertheless exhibits a transparency such that the spheres (S1) remain perfectly visible. At the time of application, actual colouring of the lips occurs, which may seem surprising, given the transparency of the composition prior to application.

In the context of the invention, and unless otherwise indicated, the term “colouring agent” or “colouration agent”, is understood to refer to a material that serves to give the composition a certain colouring, and in particular a long-lasting colour. The term “colour” is understood to encompass for example, white, black and all other colours of the visible spectrum, such as blue, red, yellow . . . possibly in iridescent, glossy form or in other known forms.

In other words, the term “colouring agent” within the meaning of the present invention, is understood to refer to a compound that is capable of producing a coloured optical effect when it is formulated in a sufficient amount in an appropriate cosmetic medium.

Within the meaning of the present invention, a composition according to the invention comprises at least one colouring agent selected from among organic or inorganic dyes and colouring agents whether or not water-soluble, whether or not liposoluble (fat-soluble), optical effect providing materials, liquid crystals, and the mixtures thereof.

The term “water-soluble colouring agent” within the meaning of the invention is understood to refer to, any compound generally organic, natural or synthetic in nature, that is soluble in an aqueous phase, or to solvents that are miscible with water and capable of imparting colour. In particular, the term ‘water-soluble’ is understood to characterise the ability of a compound to dissolve in water, measured at 25° C., at a concentration at least equal to 0.1 g/l (obtaining of a macroscopically isotropic and transparent solution, whether or not coloured). This solubility is in particular greater than or equal to 1 g/l.

A colouring agent according to the invention is preferably selected from among pigments, dyestuffs and colourings, liquid crystals and the mixtures thereof.

Preferably, the colouring agent is selected from dyestuffs and colourings.

According to the invention, the dyestuffs/colourings are typically substantially soluble in the medium of use thereof, in particular as defined in the DIN 55944 standard (December 2011).

According to one embodiment, the colouring agent according to the invention is selected from optical effect providing materials.

The particles with metallic sheen that may be used in the invention are in particular selected from among:
particles of at least one metal and/or at least one metal derivative;
particles comprising an inorganic or organic substrate, formed from single or multiple materials, coated or covered at least partially by at least one reflective metallic layer comprising at least one metal and/or at least one metal derivative; and
mixtures of the said particles.

Among the metals which may be present in the said particles, mention may be made for example, of: Ag, Au, Cu, Al, Ni, Sn, Mg, Cr, Mo, Ti, Zr, Pt, Va, Rb, W, Zn, Ge, Te, Se and mixtures or alloys thereof. Ag, Au, Cu, Al, Zn, Ni, Mo, Cr, and mixtures or alloys thereof (for example, bronzes and brasses) are preferred metals.

The term “metal compounds” is understood to refer to compounds derived from metals, in particular, oxides, fluorides, chlorides and sulfides.

According to one embodiment, the colouring agent according to the invention is selected from the liquid crystals. According to the invention, the terms “liquid crystals” or “liquid crystal type colouring agent” are understood to refer to the liquid crystals colouring the composition, that is to say providing a colouration to the composition as specified here above. Liquid crystals are typically characterised by an intermediate state between the crystalline phase, where there an order of three-dimensional position prevails and the liquid phase where no order exists.

Alternatively, the colour effect of the fatty phase can be obtained by the use in implementation of naturally coloured oil(s), such as Annatto oil, Lipocarotene, or an extract of dyers gromwell.

According to one embodiment, the aqueous phase of a composition according to the invention comprises between 0.0001% and 15% by weight of colouring agent(s), preferably from dyestuff-colouring(s), in relation to the weight of the aqueous phase.

According to one embodiment, the fatty phase of a composition according to the invention comprises between 0.0001% and 15% by weight of colouring agent(s), preferably from dyestuff-colouring(s), in relation to the weight of the fatty phase.

Preferably, a composition according to the invention comprises less than 2%, in particular less than 1%, preferably less than 0.5%, and in particular less than 0.1% by weight of pigments in relation to the total weight of the said composition.

Preferably, a composition according to the invention, in particular the fatty phase, is free of pigments.

According to one embodiment, when the aqueous phase and/or the fatty phase comprises at least one colouring agent, in particular at least one dyestuff, the said aqueous phase and/or the said fatty phase, preferably at least the fatty phase additionally also comprises UV sunscreen filters, in order to prevent/avoid undesired changes in colour.

According to one embodiment, the aqueous phase may further comprise glycerin.

Preferably, a composition according to the invention comprises at least 2%, preferably at least 5%, in particular at least 10%, in particular at least 20%, or even at least 30%, or indeed even at least 40%, or at least 50% by weight of glycerin in relation to the weight of the aqueous phase. This type of raw material serves to counter or retard the limits of the formulation without altering the finish and effect on the keratin material.

### Other Embodiments

According to one particular embodiment, the aqueous phase comprises an intermediate phase, the intermediate phase being placed in contact with the fatty phase, and at least one internal phase disposed in the intermediate phase. Such an embodiment corresponds to spheres provided with a “drop-within-drop” type architecture. In other words, the internal phase is disposed completely away from the fatty phase, the intermediate phase being interposed between the or each internal phase and the fatty phase.

The intermediate phase is thus characterised by the aqueous phase as previously described here above.

The internal phase may be hydrophilic or lipophilic in nature. The internal phase may be solid or liquid at ambient temperature and atmospheric pressure.

The internal phase may further comprise at least one gelling agent and/or any additional compound/active agent, in particular as described here above.

According to another particular embodiment:
the aqueous phase may be present in the form of a direct emulsion (oil-in-water), the said emulsion comprising a continuous aqueous phase and a dispersed fatty phase dispersed in the form of droplets (G2), the size of droplets (G2) being necessarily smaller than the size of the spheres (S1). In particular, the size of the droplets (G2) is less than 500 μm, preferably less than 400 μm, in particular less than 300 μm, more preferably less than 200 μm, in particular less than 100 μm, or indeed even less than 20 μm, and even more preferably less than 10 μm. Preferentially, the size of the droplets (G2) is comprised between 0.1 and 200 μm μm, preferably between 0.25 μm and 100 μm, in particular between 0.5 μm and 50 μm, preferably between 1 μm and 20 μm, and better still between 1 μm and 10 μm or indeed even between 3 μm and 5 μm;

and/or
the fatty phase may be present in the form of an inverse emulsion (water-in-oil), the said emulsion comprising a continuous fatty phase and a dispersed aqueous phase dispersed in the form of droplets (G3), the size of droplets (G3) being preferably microscopic. In particular, the size of droplets (G3) is less than 500 μm, preferably less than 400 μm, in particular less than 300 μm, more preferably less than 200 μm, in particular less than 100 μm, or indeed even less than 20 μm, and more preferably less than 10 μm. Preferentially, the size of droplets (G3) is comprised between 0.1 μm and 200 μm, preferably between 0.25 μm and 100 μm, in particular between 0.5 μm and 50 μm, preferably between 1 μm and 20 μm, and more preferably still between 1 μm and 10 μm or indeed even between 3 μm and 5 μm.

Optionally, the droplets (G2) and/or (G3) comprise a shell formed of at least one anionic polymer, in particular a carbomer, and at least one cationic polymer, in particular an amodimethicone, the said anionic and cationic polymers being as defined here above.

Advantageously, the droplets (G2) and/or (G3) are not macroscopic, and therefore are microscopic, that is to say, not visible to the naked eye.

In other words, the droplets (G2) and/or (G3) are different and independent of the spheres (S1).

These droplets (G2) and/or (G3) of reduced size therefore provide the means to obtain an advantageous effect on the texture. Indeed, a composition according to the invention comprising such droplets (G2) and/or (G3) finely dispersed, exhibits further improved qualities/properties of unctuousness or creaminess.

The presence of the droplets (G2) and/or (G3) enhances the characteristics of a composition according to the invention in terms of unique texture, lightness and evolving sensorial appeal. More particularly, a composition according to the invention comprising droplets (G2) and/or (G3) spread easily over a keratinous material, in particular the lips. This texture is particularly advantageous and pleasantly surprising for the person skilled in the art.

A composition according to the invention is in the main dedicated to make-up and/or care products for keratin materials, in particular the lips and/or eyelids. The person skilled in the art will know how to proceed with making adjustments in terms of nature and/or amount of raw materials in order to target the composition according to the invention towards make up or care products for keratin materials, in particular with respect to the selection of oils that may be used in effective implementation in respect of the fatty phase.

Method

The compositions according to the invention can be prepared by various methods.

With regard to the manufacturing method, a composition according to the invention is advantageously produced in a single step in that there is no preliminary step to carry out in advance for forming the spheres (S1) before mixing with the suspensive fatty phase, which is advantageous in terms of industrial scale production.

Thus, the compositions according to the invention present the advantage of being prepared according to a simple “non-microfluidic” method, that is to say by means of simple emulsification. Just as in a conventional emulsion, an aqueous solution and a fatty (or oily) solution are prepared separately.

They can also be prepared, as indicated here above, by means of a “microfluidic” method, particularly as described in the international patent applications WO 2012/120043 and WO 2015/055748, and particularly in “liquid jet” mode (referred to as: jetting) (by formation of a liquid jet at the outlet of the microfluidic device, and then fragmentation of the jet in ambient air under the effect of gravity) or drop-by-drop mode (referred to as: dripping) as described in the patent application WO 2012/120043.

In order to prepare a composition according to the invention, use is made of an internal fluid (IF), for constituting the dispersed aqueous phase, and of an external fluid (OF), for constituting the continuous fatty phase.

In view of the foregoing, the fluid (IF) comprises at least one hydrophilic gelling agent and water, and in addition, in an optional manner, at least one additional component as described here above.

The fluid (OF) includes at least one lipophilic agent having suspending capacity, preferably at least one oil, and in addition, in an optional manner, at least one additional component as described here above.

According to one embodiment, the preparation method for preparing a composition according to the invention comprises a contacting step for bringing in contact a fluid (IF) and a fluid (OF) as defined here above.

Depending on the nature and/or on the content of hydrophilic gelling agent(s) and lipophilic agent(s) having suspending capacity, the step of bringing in contact the fluids (IF) and (OF) is to be carried out with an aqueous and/or a fatty phase preheated in advance to a temperature considered adequate to ensure thereto a sufficiently fluid character to effectively enable:
a homogeneous mixture and proper formation of the spheres (S1) if the method is non-microfluidic, or
the proper formation of the spheres (S1) in the fatty phase if the method is microfluidic (with the availability as needed of a cooling device post-fabrication of the spheres (S1) in order to more rapidly solidify the spheres (S1) and thereby prevent alteration thereof).

According to yet another embodiment, when the fluid (IF) comprises at least one heat-sensitive gelling agent and/or the fluid (OF) comprises at least one lipophilic agent having a heat-sensitive suspending capacity, such as is described here above, the emulsion preparation method for preparing an emulsion according to the invention may require the use in implementation of at least the fluid (IF) and/or (OF) at a temperature ranging from 40° C. to 150° C.

Thus, according to this embodiment, the fluid (IF) and/or (OF) may be heated to a temperature in the range of 40° C. to 150° C.

In the event where the emulsion preparation method for preparing an emulsion according to the invention is a microfluidic method, the microfluidic device as such is advantageously heated to a temperature in the range of 40° C. to 150° C.

Uses

The compositions according to the invention may in particular be used in the cosmetics field.

They may comprise, in addition to the aforementioned ingredients, at least one physiologically acceptable medium.

The term “physiologically acceptable medium” is understood to refer to a medium that is particularly suitable for the applying of a composition according to the invention to the keratin materials, in particular the skin, the lips, the nails, the eyelashes, or the eyebrows, and preferably the skin.

The physiologically acceptable medium is generally adapted to the nature of the support substrate on which the composition is to be applied, as well as the aspect under which the composition is to be packaged.

According to one embodiment, the physiologically acceptable medium is the aqueous continuous phase as described here above.

According to one embodiment, the cosmetics compositions are used for make-up and/or care purposes in respect of keratin materials, in particular the skin.

The cosmetics compositions according to the invention may be products for personal care, sun protection (sunscreen), cleaning (make-up removal/cleansing), hygiene or make-up (skin).

These compositions are therefore intended to be applied in particular on the skin.

Thus, the present invention also relates to the non-therapeutic cosmetic use of a cosmetic composition as mentioned above, as a product for make-up, personal hygiene, cleaning (make-up removal/cleansing) and/or care for keratin materials, in particular the skin.

According to one embodiment, the compositions of the invention are in the form of a foundation, a makeup remover, a treatment/product for face and/or body and/or hair care, anti-aging, a sunscreen, an oily skin care, a whitening treatment, a moisturiser, a BB cream, tinted cream or make-up foundation, a face and/or body cleanser, a shower gel or a shampoo.

A care-treatment composition according to the invention may be in particular a sun protection composition, a skin cream, a serum or deodorant.

The compositions according to the invention may be in various different forms, in particular in the form of a cream, balm, lotion, serum, gel, gel-cream or even mist.

In particular, the compositions according to the invention are intended to be applied on to the lips or the eyelids.

Preferably, the compositions according to the invention are in the form of glosses (or lip gloss), lipsticks, concretes, eyeliners, or eye glosses.

The present invention also relates to a non-therapeutic method of cosmetic treatment for treating a keratin material, in particular the lips and/or the eyelids, comprising at least one application step of applying on to the said keratin material at least one composition as defined here above.

In particular, the present invention relates to a non-therapeutic method of cosmetic treatment for treating the skin, in particular the lips and/or the eyelids, comprising one application step of applying on to the skin at least one layer of a cosmetic composition as defined here above.

Throughout the description, the term “comprising one/a” is to be understood as being synonymous with “comprising at least one”, unless otherwise specified.

The terms “comprised between . . . and . . . ”, “in the range/ranging from . . . to . . . ”, and “going from . . . to . . . ”, are to be understood as being inclusive of limits, unless otherwise specified.

The amounts of the ingredients listed in the examples are expressed in terms of percentages by weight in relation to the total weight of the composition, unless otherwise indicated.

The examples that follow Illustrate the present invention without intending to limit the scope thereof.

### EXAMPLES

### Example 1: Preparation of an Eye Gloss by a Non Microfluidic Method

The table below indicates the ingredients of the final composition (eye gloss) as well as the nature of the different phases that were operationally implemented.

Final Composition

| | | | % w/w | % w/w | |
|---|---|---|---|---|---|
| Name | Supplier | INCI Name | Phases | final | Phase |
| AQUEOUS PHASE (= Spheres (S1)) sub total | | | 100.00 | 15.000 | |
| Reverse osmosis | / | Aqua | qsp | qsp | A1 |
| Water | | | | | |
| Microcare PE | Thor | Phenoxyethanol, aqua | 0.80 | 0.120 | A1 |
| Microcare | Thor | Pentylene Glycol, aqua | 2.00 | 0.300 | A1 |
| Emollient PTG | | | | | |
| Carbopol Ultrez 10 | Lubrizol | CARBOMER | 0.30 | 0.045 | A2 |
| Agar | VAHINE | Agar Agar | 0.30 | 0.045 | A1 |
| Glycerine codex | INTERCHIMIE | Glycerin | 53.33 | 8.000 | A3 |
| (99%) | | | | | |
| Crystalhyal 1.0 | | Sodium hyaluronate | 0.07 | 0.010 | A3 |
| EDETA BD | BASF | DISODIUM EDTA | 0.04 | 0.006 | A1 |
| Sodium Hydroxide | Panréac | SODIUM HYDROXIDE | 0.05 | 0.008 | A3 |
| Pellets PRS | | | | | |
| Codex | | | | | |
| OILY PHASE sub total | | | 100.00 | 85.000 | |
| Dub Inin Grade A | Stéarinerie | Isononyl Isononate | qs | qsp | B1 |
| | Dubois | | | | |
| Polybut 10 | INTERCHIMIE | Polybutene | 37.65 | 32.000 | B2 |
| ASL-1 RED R-516P | Daito Kasei | CI 77491 (and) Sodium | 0.34 | 0.288 | B2 |
| | | Dilauramidoglutamide Lysine | | | |
| | | (and) Magnesium Chloride | | | |
| Covalumine Triple | Sensient | CI 77499 (and) Alumina | 1.01 | 0.856 | B2 |
| Black AS | | (and) triethoxycaprylylsilane | | | |
| Aerosil R812 | Evonik | Silica Silyate | 5.88 | 5.000 | B2 |
| FA4002 ID | Dow Corning | Isododecane (and) Acrylates/ | 11.76 | 10.000 | C |
| | | Polytrimethylsiloxy- | | | |
| | | methacrylate Copolymer | | | |
| CAS-3131 PILOT | Nusil | AMODIMETHICONE | 0.118 | 0.100 | B1 |

| TOTAL | | | 100.000 |
|---|---|---|---|
| Manufacture | Sodium Hydroxide | 0.5000 | 0.0750 |
| solution | | | |
| soda 10% | | | |

Preparation of the Aqueous Phase (IF)

a) The A1s are mixed together under agitation at 85° C. until a homogeneous mixture is obtained;

b) A2 is added to the mixture a) without agitation which then is left to stand for a period of 15 minutes until hydration of B2 occurs; then, agitation is carried out until a homogeneous mixture is obtained;

c) A3 is added to the mixture b) under agitation until a homogeneous mixture is obtained, in a manner so as to obtain the aqueous phase.

Preparation of the Oily Phase (OF)

a) All the B1s are mixed together under agitation until a homogeneous mixture is obtained; and

b) All the B2s are added therein until a homogeneous mixture is obtained, in a manner so as to obtain the oily OF.

Preparation of the Composition According to the Invention

a) The oily OF is available for use at 85° C. and the aqueous IF is available for use at 85° C.;

b) The aqueous IF at 85° C. Is added Into the oily OF at 85° C. under agitation; and

c) when the mixture b) is at 40° C., the C is added therein under agitation.

The eye gloss according to Example 1 exhibits both a high degree of glossy lustre and particularly satisfactory capacities in respect of hydration, freshness and comfort on application. This satisfactory degree of glossy lustre is accompanied by good performance and staying power/durability over time with the sensation upon application being acceptable in terms of tackiness (stickiness) and any resultant impedance to comfort and ease of use.

### Examples 2 to 5: Preparation of Cosmetic Compositions According to the Invention

The composition according to Example 2 is a transparent formula for lip gloss obtained by means of a microfluidic method implemented in dripping mode.

The composition according to Example 3 is a fatty phase-based formula for coloured lip gloss and obtained by means of a microfluidic method in dripping mode.

The composition according to Example 4 is an aqueous phase-based formula for coloured lip gloss and obtained by means of a microfluidic method in dripping mode.

The composition according to Example 5 is a formula for lip gloss obtained by means of a microfluidic method in jetting mode.

The phases used to prepare these formulations are as follows:

Aqueous Phase (IF)

| | | | Ex 2 | Ex 3 | Ex 4 | Ex 5 and 6 | |
|---|---|---|---|---|---|---|---|
| Name | Supplier | INCI name | % w/w | % w/ w | % w/w | % w/w | |
| Reverse Osmosis | / | Aqua | qsp | qsp | qsp | qsp | B1 |
| Water | | | | | | | |
| Microcare PE | THOR | Phenoxyethanol | 0.80 | 0.80 | 0.80 | 0.80 | B1 |
| Microcare PTG | THOR | Pentylenglycol | 2.00 | 2.00 | 2.00 | 2.00 | B1 |
| Carbopol ULTREZ 10 | Lubrizol | Carbomer | 0.20 | 0.20 | 0.20 | 0.20 | B2 |
| Glycerine codex (99%) | INTERCHIMIE | Glycerin | 8.00 | 7.00 | 15.00 | 17.00 | B3 |
| Crystalhyal 1.0 | — | Sodium hyaluronate | 1.00 | 1.00 | 1.00 | 1.00 | B3 |
| Agar | VAHINE | Agar Agar | 0.50 | 0.48 | 0.90 | 0.55 | B3 |
| Sodium Hydroxide | PANREAC | Sodium Hydroxide | 0.033 | 0.033 | 0.033 | 0.33 | B3 |
| 10% solution | | | | | | | |
| Unicert Red K7057 J | SENSIENT | CI17200 | 0.00 | 0.00 | 0.03 | 0.00 | B4 |

| | | | | |
|---|---|---|---|---|
| Total | 100.00 | 100.00 | 100.00 | 100.00 |

Oily Phase (OF)

| | | | Ex 2 | Ex 3 | Ex 4 | Ex 5 | Ex 6 | |
|---|---|---|---|---|---|---|---|---|
| Name | Supplier | INCI Name | % w/w | % w/w | % w/w | % w/w | % w/w | |
| Parleam | ROSSOW | Hydrogenated Polybutene | 28.88 | 28.88 | qs | 26.50 | 0.00 | A1 |
| Cyclomethicone 5- NF | Dow Corning | Cyclopentasiloxane | 0.00 | 0.00 | 0.00 | 0.00 | qsp | A1 |
| | Corporation | | | | | | | |
| CareSilTM CXG-1104 | NUSIL | Dimethicone (and) | 0.00 | 0.00 | 0.00 | 0.00 | 14.00 | A1 |
| | | Dimethicone/Vinyl Dimethicone | | | | | | |
| | | Crosspolymer | | | | | | |
| Salacos 222 | SACI CFPA | Diisostearyl malate | 31.40 | 26.90 | 0.00 | qs | 0.00 | A1 |
| Polybutene | INTERCHIMIE | Polybutene | qs | qs | 48.45 | 35 | 0.00 | A1 |
| Aerosil R974 | EVONIK | Silica DimethylSilylate | 4.00 | 4.00 | 3.00 | 300 | 0.00 | A2 |
| VM-2270 | Dow Corning | Silica silylate | 0.00 | 0.00 | 0.00 | 0.00 | 0.90 | A2 |
| Lipocarotte CLR | CLR | Glycine Soja (Soybean) oil, | 0.00 | 4.00 | 0.00 | 0.00 | 0.00 | A1 |
| | | Daucus carota extract beta | | | | | | |
| | | carotene tocopherol | | | | | | |
| Covabsorb | SENSIENT | Ethylhexyl methoxycinnamate | 0.00 | 0.50 | 0.00 | 0.00 | 0.00 | A1 |
| | | and butyl | | | | | | |
| | | methoxydibenzoylmethane and | | | | | | |
| | | ethylhexylsalicilate | | | | | | |
| Vitamin E | BASF | Tocopheryl acetate | 0.00 | 0.00 | 0.00 | 0.50 | 0.00 | A3 |

| | | | | | |
|---|---|---|---|---|---|
| Total | 100.00 | 100.00 | 100.00 | 100.00 | 100.00 |

Preparation of the Aqueous Phase (IF)

a) All the B1s are mixed together under agitation until a homogeneous mixture is obtained;

b) All the B2s are added to the mixture a) without agitation which then is left to stand for a period of 15 minutes until hydration of B2 occurs; then, agitation is carried out until a homogeneous mixture is obtained;

c) in parallel, a mixture is prepared with all the B3s at 90° C. until a homogeneous mixture is obtained in liquid form (molten);

d) the mixture c) is added to the mixture b), under agitation at 90° C., until a homogeneous mixture is obtained, in a manner so as to obtain the aqueous phase IF; and

e) when present, all the B4s are added to the mixture d).

Preparation of the Oily Phase (OF)

a) The A1s are mixed together under agitation until a homogeneous mixture is obtained;

b) A2 is added to the mixture a) under agitation, with agitation then being carried out until a homogeneous mixture is obtained; and

c) when present, all the A3s are added therein under agitation until a homogeneous mixture is obtained, in a manner so as to obtain the oily OF.

Preparation of the Compositions

The compositions according to Examples 2 to 5 are obtained in accordance with a microfluidic method, that is to say using a microfluidic nozzle with double casing envelope (pipe-in-pipe) construction as described in the document WO2012/120043, the inner diameter of the outlet of the nozzle is 0.8 mm.

At the level of the microfluidic device, the parameters/settings are as follows:

| | Ex 2 | Ex 3 | Ex 4 | Ex 5 | Ex 6 |
|---|---|---|---|---|---|
| OF Flow rate | 100 | 100 | 80 | 300 | 100 |
| (in mL/h/nozzle) | | | | | |
| IF Flow Rate | 5 | 5 | 4 | 35 | 3 |
| (in mL/hr/nozzle) | | | | | |
| T° C. OF | TA | TA | TA | TA | TA |
| T° C. IF | 85 | 90 | 85 | 90 | 80% |
| IF in the final | 4.76 | 4.76 | 4.76 | 9.1 | 2.91% |
| composition | | | | | |
| OF in the final | 95.24 | 95.24 | 95.24 | 90.9 | 97.09 |
| composition | | | | | |
| Notes | Dripp- | Dripp- | Dripp- | Jett- | Dripp- |
| | ing | ing | ing | ing | ing |

In addition to a unique visual effect linked to the presence of macroscopic aqueous bubbles in the oily continuous phase and the transparency/translucency of the compositions, these compositions for lip glosses exhibit both a high degree of glossy lustre and particularly satisfactory capacities in respect of hydration, freshness and comfort on application. This satisfactory degree of glossy lustre is accompanied by good performance and staying power (durability) over time without the sensation of tackiness (stickiness) upon application nor any resultant impedance to comfort and ease of use.

Finally, the composition according to Example 6 exhibits an oily continuous phase with satisfactory transparency properties.

### Example 7: Preparation of a Perfumed Concrete by a Microfluidic Method in Dripping Mode

The phases used to prepare this formulation are as follows:

Aqueous Phase (IF)

| | | INCI | | |
|---|---|---|---|---|
| Name | Supplier | Name | % w/w | |
| Reverse Osmosis | / | Aqua | qs | B1 |
| Water | | | | |
| Carbopol ULTREZ 10 | Lubrizol | Carbomer | 0.1 | B2 |
| Microcare PE | THOR | Phenoxyethanol | 0.8 | B1 |
| Microcare Emollient PTG | THOR | Pentylenglycol | 2 | B1 |
| Orange blossom water | | | 9.94 | B3 |
| Soda 10% | | | 0.12 | B3 |
| Agar Agar | Vahine | Agar Agar | 0.4 | B3 |
| Dye solution 0.5% | | | 7.93 | B3 |

| | |
|---|---|
| Total | 100.00 |

Oily Phase (OF)

| | | INCI | | |
|---|---|---|---|---|
| Name | Supplier | Name | % w/w | |
| Dimethicone | Shin Etsu | Dimethicone | 9.0 | A1 |
| KF 96A 6 cts | | | | |
| CAS 3131 | NUSIL | Amodimethicone | 0.5 | A1 |
| Dub Inin | Stéarinerie | Isononyl | qs | A1 |
| | Dubois | Isononate | | |
| Floraesters | Floratech | Jojoba Esters | 34.6 | A2 |
| 30 | | | | |
| Bees Wax | — | — | 23.5 | A2 |

| | |
|---|---|
| Total | 100.00 |

Preparation of the Aqueous Phase (IF)

a) All the B1s are mixed together under agitation until a homogeneous mixture is obtained;

b) All the B2s are added to the mixture a) without agitation which then is left to stand for a period of 15 minutes until hydration of B2 occurs; then, agitation is carried out until a homogeneous mixture is obtained;

c) in parallel, a mixture is prepared with all the B3s at 90° C. until a homogeneous mixture is obtained in liquid form (molten);

d) the mixture c) is added to the mixture b), under agitation at 90° C., until a homogeneous mixture is obtained, in a manner so as to obtain the aqueous phase IF.

Preparation of the Oily Phase (OF)

a) The A1s are mixed together under agitation until a homogeneous mixture is obtained;

b) on the one hand, the mixture a) is heated to 80° C. and, on the other hand, all the A2s are heated to 80° C.; and

c) the A2s in liquid form (molten) are added to the mixture a), until a homogeneous mixture is obtained, in a manner so as to obtain the oily OF.

Preparation of the Composition According to the Invention

The composition according to Example 7 is obtained in accordance with a microfluidic method, that is to say using a microfluidic nozzle with double casing envelope (pipe-in-pipe) construction as described in the document WO2012/120043, the inner diameter of the outlet of the nozzle is 0.8 mm.

The parameters/settings are as follows:

| | |
|---|---|
| OF Flow rate | 100 |
| (in mL/h/nozzle) | |
| IF Flow rate | 7.5 |
| (in mL/h/nozzle) | |
| T° C. OF | 80 |
| T° C. IF | 90 |

The composition according to Example 7 is present in the form of a concrete of perfume at ambient temperature (AT).

In addition to a unique visual effect linked to the presence of macroscopic aqueous bubbles in the oily continuous phase, this composition has very significant perfuming power satisfactorily complemented with appealing sensorial properties.

### Example 8: Preparation of a Cosmetic Composition (Serum) by a Microfluidic Method at Ambient Temperature

The phases used to prepare this formulation are as follows:

Aqueous Phase (IF)

| | | INCI | | |
|---|---|---|---|---|
| Name | Supplier | name | % w/w | |
| Reverse osmosis | / | Aqua | Qsp * | B1 |
| water | | | | |
| Microcare PE | THOR | Phenoxyethanol | 0.80 | B1 |
| Microcare PTG | THOR | Pentylenglycol | 2.00 | B1 |
| EDETA BD | BASF | Disodium EDTA | 0.03 | B1 |
| Glycerine codex | INTERCHIMIE | Glycerin | 2.00 | B4 |
| (99%) | | | | |
| ZEMEA | DUPONT TATE | Propanediol | 3.00 | B4 |
| PROPANEDIOL | & LYLE | | | |
| Alcasealan | HAKUTO | Alcaligenes | 0.02 | B2 |
| | | Polysaccharides | | |
| Carbopol ETD 2050 | LUBRIZOL | Carbomer | 0.10 | B3 |
| Sodium Hydroxide | PANREAC | Sodium | 0.02 | B5 |
| Pellets PRS codex | | Hydroxide | | |

| | |
|---|---|
| Total | 100 |

* Sufficient Quantity For [Quantité Suffisante Pour]

Oily Phase (OF)

| Name | Supplier | INCI Name | % w/w | |
|---|---|---|---|---|
| DUB ININ Grade A | STEARINERIE | Isononyl isononanoate | Qsp | A1/A2 |
| | DUBOIS | | | |
| Estogel M | POLYMER | CASTOR OIL/IPDI | 2.50 | A1 |
| | EXPERT | COPOLYMER & | | |
| | | CAPRYLIC/CAPRIC | | |
| | | TRIGLYCERIDE | | |
| DUB SSIC MB | STEARINERY | Isocetyl Stearoyl | 7.50 | A3 |
| | DUBOIS | Stearate | | |
| DUB 810C | STEA RINERIE | Coco-Caprylate/ | 10.00 | A3 |
| | DUBOIS | Caprate | | |
| Meadowfoam Oil | NIKKOL | Limnanthes Alba | 7.50 | A3 |
| | CHEMICALS | (Meadowfoam) Seed Oil | | |
| CAS 3131 | NUSIL | Amodimethiconeflow | 0.30 | A2 |

| | |
|---|---|
| Total | 100 |

Preparation of the Aqueous Phase (IF)

a) All the B1s are mixed together under agitation until a homogeneous mixture is obtained;

b) All the B2s are added to the mixture a) under agitation using the Silverson (2500 rpm) device until a homogeneous mixture is obtained (about 15 minutes);

c) then B3 is added therein without agitation and the mixture is left to stand so as to hydrate B3 (about 15 minutes); thereafter agitation is carried using the Rayneri apparatus until a homogeneous mixture is obtained,

d) All the B4s are added to the mixture c) under agitation until a homogeneous mixture is obtained; and

e) the B5 is added to the mixture d) under agitation until a homogeneous mixture is obtained.

Preparation of the Oily phase (OF)

a) The A1s are mixed together under very vigorous agitation at 95° C. for a period of 1 hour;

b) In parallel, all the A2s are mixed therein under agitation at 80° C.,

b) The mixture b) is added to the mixture a) and then, agitation is carried out at 80° C. until a homogeneous mixture is obtained,

c) all the A3s are added to the mixture b), until a homogeneous mixture is obtained; and

d) the whole mixture is then allowed to cool until it returns to ambient temperature, following which it may then be used to form the composition according to example 8.

Preparation of the Composition According to the Invention

The composition according to Example 8 is obtained in accordance with a microfluidic method, that is to say using a microfluidic nozzle with double casing envelope (pipe-in-pipe) construction as described in the document WO2012/120043.

The parameters/settings are as follows:

| | |
|---|---|
| OF Flow rate | 129 |
| (in mL/h/nozzle) | |
| IF Flow Rate | 3 |
| (in mL/hr/nozzle) | |
| T ° C. OF | TA * |
| T ° C. IF | TA * |

* TA = Ambient Temperature

The composition according to Example 8 is in the form of a serum at ambient temperature (TA).

In addition to a unique visual effect linked to the presence of macroscopic aqueous bubbles in the oily continuous phase, the said composition exhibits both a satisfactory texture and appealing sensorial properties.

A composition according to Example 8 was also produced without amodimethicone in the oily phase; it yielded satisfactory visual effect, and texture accompanied by appealing sensorial properties.

What is claimed is:

1. A composition, comprising a fatty phase and an aqueous phase, wherein:
the aqueous phase is in the form of spheres (S1) that are solid at ambient temperature and atmospheric pressure, comprising at least one hydrophilic gelling agent; and
the fatty phase comprises at least one lipophilic agent having suspending capacity.

2. The composition according to claim 1, wherein the hydrophilic gelling agent is selected from the group consisting of natural texturing agents, semi-synthetic texturing agents, synthetic texturing agents, and the mixtures thereof.

3. The composition according to claim 1, wherein the hydrophilic gelling agent is selected from natural texturing agents that are temperature-sensitive.

4. The composition according to claim 1, wherein the content of hydrophilic gelling agent(s) is comprised between 0.1% and 15% by weight in relation to the weight of aqueous phase of the said composition.

5. The composition according to claim 1, wherein the content of hydrophilic gelling agent(s) is comprised between 0.5% and 0.9% by weight in relation to the weight of aqueous phase of the said composition.

6. The composition according to claim 1, wherein the aqueous phase comprises at least two hydrophilic gelling agents, at least one being a temperature-sensitive hydrophilic gelling agent.

7. The composition according to claim 1, wherein the content of temperature-sensitive hydrophilic gelling agent(s), is comprised between 0.1% and 15% by weight in relation to the weight of the aqueous phase of the said composition.

8. The composition according to claim 1, wherein the content by weight of aqueous phase is comprised between 1% and 30% by weight in relation to the total weight of the said composition.

9. The composition according to claim 1, wherein the fatty phase content is comprised between 70% and 99% by weight in relation to the total weight of the said composition.

10. The composition according to claim 1, wherein the lipophilic gelling agent having suspending capacity is selected from among lipophilic gelling agents, either organic or inorganic, polymeric or molecular; solid fatty substances at ambient temperature and pressure; and the mixtures thereof.

11. The composition according to claim 1, wherein the lipophilic agent having suspending capacity is selected from silicas and hydrophobic silica aerogels.

12. The composition according to claim 1, wherein the content of lipophilic agent(s) having suspending capacity is comprised between 0.5% and 99.50% by weight in relation to the total weight of the fatty phase, or indeed relative to the total weight of the composition.

13. The composition according to claim 1, wherein the fatty phase comprises at least one oil.

14. The composition according to claim 1 comprising at least one colouring agent in the aqueous phase and/or the fatty phase.

15. The composition according to claim 1, characterised in that it does not include a surfactant.

16. A non-therapeutic method of cosmetic treatment for treating a keratin material, comprising at least one application step of applying on to the said keratin material at least one composition according to claim 1.
